SEQUENCE LISTING (1 sequence)

SEQ ID NO: 1 - DNA, 20 bp, synthetic construct
  source 1..20: mol_type = other DNA
acaaaaggcctccttcatta

(19) United States
(12) Patent Application Publication
(10) Pub. No.: US 2024/0327909 A1
(43) Pub. Date: Oct. 3, 2024

(54) COMPOSITIONS AND METHODS FOR NUCLEIC ACID SEQUENCING

(71) Applicant: Illumina, Inc., San Diego, CA (US)

(72) Inventors: Xiaolin Wu, Cambridge (GB); Pietro Gatti Lafranconi, Cambridge (GB); Timothy Beech, Cambridge (GB); Carlo Bravin, Cambridge (GB); Benedict Mackworth, Cambridge (GB); Thomas Tongue, Cambridge (GB); Philip Balding, Cambridge (GB)

(21) Appl. No.: 18/618,263

(22) Filed: Mar. 27, 2024

Related U.S. Application Data
(60) Provisional application No. 63/493,187, filed on Mar. 30, 2023.

(51) Int. Cl. C12Q 1/6874; G01N 33/58

(52) U.S. Cl. CPC C12Q 1/6874; G01N 33/582

(57) ABSTRACT

Embodiments of the present application relates to methods and compositions for increasing fluorescent dye signal intensity during sequencing by synthesis. In particular, the compositions and methods described herein involve the use aqueous scan mixtures including one or more water-soluble macrocycles that can form host-guest complexes with the dyes.

### CROSS-REFERENCE TO RELATED APPLICATION

This application claims the benefit of priority to U.S. Provisional Application No. 63/493,187, filed Mar. 30, 2023, the content of which is incorporated by reference in its entirety.

### BACKGROUND

### Field

The present disclosure generally relates to polynucleotide sequencing methods, compositions, and kits for sequencing.

### REFERENCE TO SEQUENCE LISTING

The present application is being filed along with a Sequence Listing in electronic format. The Sequence Listing is entitled Sequence_Listing_ILLINC762A.xml created on Mar. 21, 2024, which is 1,969 bytes in size. The information in the electronic format of the Sequence Listing is incorporated herein by reference in its entirety.

### DESCRIPTION OF THE RELATED ART

Non-radioactive detection of nucleic acids utilizing fluorescent labels is an important technology in molecular biology. Many procedures employed in recombinant DNA technology previously relied on the use of nucleotides or polynucleotides radioactively labeled with, for example 32P. Radioactive compounds permit sensitive detection of nucleic acids and other molecules of interest. However, there are serious limitations in the use of radioactive isotopes such as their expense, limited shelf life and more importantly safety considerations. Eliminating the need for radioactive labels enhances safety whilst reducing the environmental impact and costs associated with, for example, reagent disposal. Methods amenable to non-radioactive fluorescent detection include by way of non-limiting example, automated DNA sequencing, hybridization methods, real-time detection of polymerase-chain-reaction products and immunoassays.

For many applications it is desirable to employ multiple spectrally distinguishable fluorescent labels in order to achieve independent detection of a plurality of spatially overlapping analytes. In such multiplex methods the number of reaction vessels may be reduced to simplify experimental protocols and facilitate the production of application-specific reagent kits. In multi-color automated DNA sequencing systems for example, multiplex fluorescent detection allows for the analysis of multiple nucleotide bases in a single electrophoresis lane, thereby increasing throughput over single-color methods, and reducing uncertainties associated with inter-lane electrophoretic mobility variations.

For high-accuracy fluorescence identification of nucleobases, scanning of fluorescently labeled nucleotides under intensive expose to light is typically involved. Extensive laser irradiation, however, may bleach fluorescent dyes and/or damage nucleotide samples in solution/on flow-cell surface or those to which the fluorescent dyes are conjugated. Such expose to light may also cause DNA sample damage. Thus, there is a need particularly in multiplex fluorescent DNA sequencing to protect fluorescent dyes from photo-bleaching and polynucleotides from photo induced damages. The type and extent of photo-bleaching and photo-damages may vary depending on, for example, compound chemical structures and some their physical-chemical properties like redox potential, excitation spectra of particular bio-label, intensity of particular light source irradiation, and time of exposure in particular measurement. Since lower wavelength light sources are delivering higher energy photons, blue LED/laser having short (400-500 nm) wavelength emission (e.g., 450-460 nm) are more likely to cause photo-bleaching of dyes and associated with light DNA damage.

Performing fluorescent detection steps in an array context, such as sequencing by synthesis, can cause fluorescence signal intensity loss. The possible mechanisms that underlie this signal loss are numerous and can include cleavage of individual nucleic acid units from the solid support. There are also a number of chemical pathways by which nucleic acid damage can occur during irradiation in fluorescence detection, for example, caused by light induced reactive species, for example, Reactive Oxygen Species (ROS) such as singlet oxygen, superoxide anion, and hydroxyl radical, which may be the underlying causes of fluorescence signal intensity loss observed in the array context.

Macrocycles can enhance dye brightness, photostability, fluorescence lifetime, and protect the dye from intermolecular quenchers as described in Roy N. Dsouza et al., “Fluorescent dyes and their supramolecular host/guest complexes with macrocycles in aqueous solution,” 111 Chem. Rev. 12, 7941-80 (2011); Jyotirmayee Mohanty et al., “Ultrastable rhodamine with cucurbituril,” 44 Angewandte Chemie Int. 24, 3750-54 (2005); and Cesar Marquez et al., “Cucurbiturils: molecular nanocapsules for time-resolved fluorescence-based assays,” 3 IEEE Transactions on NanoBioscience 1, 39-45 (2004). However, use of macrocycles to enhance fluorescence intensity have not previously been reported in the context of sequencing. Described herein in are compositions and kits for improving signal intensity in nucleic acid sequencing.

### SUMMARY

One aspect of the present disclosure relates to a method of sequencing a plurality of different target polynucleotides, comprising:
(a) contacting a solid support with an incorporation mixture comprising DNA polymerase and one or more of four different types of nucleotides, wherein the solid support comprises a plurality of different target polynucleotides immobilized thereon, and sequencing primers that are complementary and hybridized to at least a portion of the target polynucleotides;
(b) incorporating one type of nucleotides into the sequencing primers to produce extended copy polynucleotides, wherein one or more of the four types of nucleotides comprises a detectable label; and
(c) imaging and performing one or more fluorescent measurements of the extended copy polynucleotides in an aqueous scan mixture;
wherein the aqueous scan mixture comprises one or more additives for improving fluorescent signal intensity of the detectable label, and wherein the one or more additives comprise one or more water-soluble macrocycles.

One aspect of the present disclosure relates to a method for enhancing the fluorescence of a fluorescent dye, comprising:
contacting the fluorescent dye with an aqueous scan mixture composition comprising one or more water-soluble macrocycles, wherein the water-soluble macrocycle forms a host-guest complex with the fluorescent dye.

Another aspect of the present disclosure relates to a kit for use with a sequencing apparatus, comprising:
an incorporation mixture comprising one or more different types of nucleotides, wherein at least one nucleotide is labeled with a detectable label;
an aqueous scan mixture comprising one or more water-soluble macrocycles, wherein the water-soluble macrocycle is capable of encapsulating the detectable label to form a host-guest complex with the detectable label.

### BRIEF DESCRIPTION OF THE DRAWINGS

FIG. 1 is a scheme illustrating encapsulation of a dye moiety by a macrocycle.

FIG. 2A is a bar chart showing normalized percentage change in fluorescence and UV-Vis absorbance of a fully functionalized C nucleotide (ffC) labeled with a coumarin blue dye (ffC-LN3-coumarin dye A) in a Tris buffer solution in the presence of various macrocycles.

FIG. 2B is a bar chart showing normalized percentage change in fluorescence absorbance of ffC-LN3-coumarin dye A in a Tris buffer solution and in a scan mixture in the presence of various macrocycles.

FIG. 3A is a bar chart showing estimated binding affinity constant (Kb) of several different types of cyclodextrin macrocycles to ffC-LN3-coumarin dye A.

FIG. 3B is a bar chart showing estimated binding affinity constant (Kb) of several calixarene macrocycles to ffC-LN3-coumarin dye A.

FIG. 3C is a bar chart showing estimated binding affinity constant (Kb) of several cucurbituril macrocycles to ffC-LN3-coumarin dye A.

FIG. 3D is a plot showing macrocycle affinity constant (Kb) to ffC-LN3-coumarin dye A in a scan mixture versus macrocycle affinity constant (Kb) to ffC-LN3-coumarin dye A in a Tris buffer.

FIGS. 4A and 4B are bar charts showing bleaching rate and normalized bleaching rate respectively for three different macrocycles.

FIGS. 4C and 4D are bar charts showing singlet oxygen production rate and normalized singlet oxygen production rate respectively for three different macrocycles.

FIG. 5A shows blue/green signal intensity and signal decay of three sequencing runs conducted on Illumina's NextSeq™ 2000 Blue/Green two-channel platform, in which the run conducted with a standard scan mixture is compared to sequencing runs conducted with hydroxypropyl-β-cyclodextrin or methyl-β-cyclodextrin added scan mixtures.

FIG. 5B shows blue/green signal intensity of two sequencing runs conducted on Illumina's NextSeq™ 2000 Blue/Green two-channel platform, in which the run conducted with a s second standard scan mixture is compared to sequencing runs conducted with methyl-β-cyclodextrin added scan mixtures.

FIGS. 6A and 6B each shows percentage increase in fluorescence intensity as a function of methyl-β-cyclodextrin concentration for ffC-sPA-LN3-coumarin dye A and ffA-LN3-BL-coumarin dye A respectively.

FIGS. 7A and 7B each shows percentage increase in fluorescence intensity as a function of methyl-β-cyclodextrin concentration for ffA-DB-AOL-BL-coumarin dye A and ffC-DB-AOL-coumarin dye A, respectively, at three different scanning temperatures.

### DETAILED DESCRIPTION

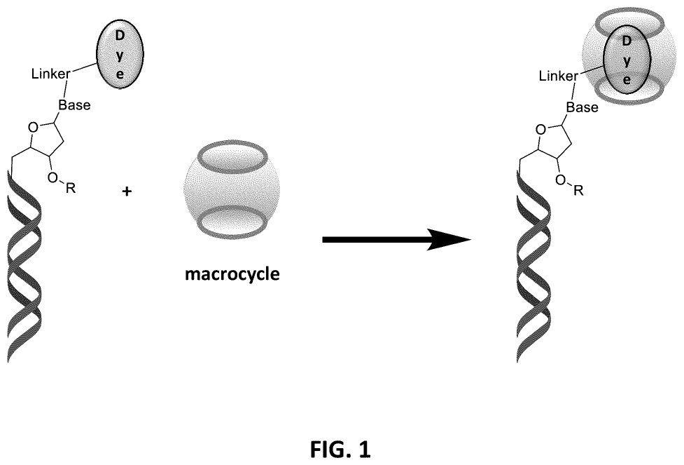

Some aspects of the present disclosure relate to kits and methods for enhancing fluorescence intensity during sequencing, and methods of protecting fluorescent dyes from quenching in aqueous solution, and thereby increase signal intensity of the dye. In particular, the compositions and methods described herein involve the use aqueous scan mixtures including one or more water-soluble macrocycles (e.g., cyclodextrins, calixarenes, or cucurbiturils, or optionally substituted analogs, salts or hydrates thereof) as additives which are capable of encapsulating the dye by forming host-guest complexes with the dye, thereby protecting the dyes and/or dye moieties from quenching (e.g., solvent induced quenching, intermolecular quenching and/or quenching by singlet oxygen), as illustrated in FIG. 1. Without being bound by a particular theory, due to the encapsulation effect, the macrocycles provide a local hydrophobic environment, which allow the enhancement of the dye brightness since non radiative pathways are disfavored. In addition, the functions of the macrocycles are attributed to protect the dye from inter molecular quenchers, possibly preventing generation of reactive oxygen species (ROS), which cause DNA damage and signal decay.

### Definitions

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
ffA Fully functionalized “A” nucleotide
ffC Fully functionalized “C” nucleotide
ffT Fully functionalized “T” nucleotide
ffG Fully functionalized “G” nucleotide
IMX Incorporation mix or Incorporation mixture
RT Room temperature
SBS Sequencing by Synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in accordance with the present disclosure to distinguish beads in a liquid array, for example, using a microfluidic device, such as a fluorescent activated cell sorter (FACS), are described, for example, in U.S. Pat. No. 6,524,793. Further examples of arrays that can be used in accordance with the present disclosure include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term quality score (“Q score”) refers to the probability that a base is called incorrectly in a nucleotide sequencing read. Q is defined as −10 log 10 (e). Higher Q scores indicate a smaller probability of error than lower Q scores. For instance, a score of Q10 indicates a 1 in 10 probability of an incorrect base call, or an inferred base call accuracy of 90%. For instance, a score of Q20 indicates a 1 in 100 probability of an incorrect base call, or an inferred base call accuracy of 99%. For instance, a score of Q30 indicates a 1 in 1,000 probability of an incorrect base call, or an inferred base call accuracy of 99.9%.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, any “R” group(s) represent substituents that can be attached to the indicated atom. An R group may be substituted or unsubstituted.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH (CH₃) CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Ca to Cb,” “Ca-Cb,” or “Ca-b” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b,” inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH (CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and Co alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁-C₉ alkoxy,” including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂.C₆ alkenyl” or similar designations. By way of example only, “C₂.C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro [4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl) alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl) alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl) alkyl” refer to a heterocyclic or a heterocyclyl group, as defined above, connected, as a substituent, via an alkylene group, as defined above. The alkylene and heterocyclyl groups of a (heterocyclyl) alkyl may be substituted or unsubstituted. Examples include but are not limited to (tetrahydro-2H-pyran-4-yl) methyl, (piperidin-4-yl) ethyl, (piperidin-4-yl) propyl, (tetrahydro-2H-thiopyran-4-yl) methyl, and (1,3-thiazinan-4-yl) methyl. In some embodiments, the alkylene is an unsubstituted straight chain containing 1, 2, 3, 4, 5, or 6 methylene unit(s).

As used herein, “(carbocyclyl) alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy) alkyl” refers to an alkoxy group connected via an alkylene group, such as C₂-C₈ alkoxyalkyl, or (C₁-C₆ alkoxy) C₁-C₆ alkyl, for example, —(CH₂)₁₋₃—OCH₃.

As used herein, “—O-alkoxyalkyl” or “—O-(alkoxy) alkyl” refers to an alkoxy group connected via an —O-(alkylene) group, such as —O—(C₁-C₆ alkoxy) C₁-C₆ alkyl, for example, —O—(CH₂)₁₋₃—OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atoms is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl (alkyl), as defined herein.

An “O-carboxy” group refers to a “—OC(═O) R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O) OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O) OH).

A “carboxylate” group refers to an “—C(═O)O⁻” group.

A “sulfonyl” group refers to an “—SO₂R” group in which R is selected from hydrogen, C₁-C₆ alkyl, C₂-C₆ alkenyl, C₂.C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O) OH” group.

A “sulfo” group refers to a “—S(═O) 2OH” or “—SO₃H” group.

A “sulfonate” group refers to a “—SO₃⁻” group.

A “sulfate” group refers to “—SO₄⁻” group.

A “S-sulfonamido” group refers to a “—SO₂NRARB” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A}) SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O) NRARB” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A}) C(═O) R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(RARB)” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O-thiocarbamyl” group refers to a “—OC(═S)—N(RARB)” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “alkylamino” or “(alkyl) amino” refers to an amino group wherein one or both hydrogen is replaced by an alkyl group.

An “(alkoxy) alkyl” group refers to an alkoxy group connected via an alkylene group, such as a “(C₁-C₆ alkoxy) C₁-C₆ alkyl” and the like.

The term “hydroxy” as used herein refers to a —OH group.

The term “cyano” group as used herein refers to a “—CN” group.

The term “azido” as used herein refers to a —N₃ group.

The term “succinyl” as used herein refers to a —C(═O) CH₂CH₂C(═O) OH group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-Cycarbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl) C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl) C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy) C₁-C₆ alkyl, —O(C₁-C₆ alkoxy) C₁-C₆ alkyl; (C₁-C₆ haloalkoxy) C₁-C₆ alkyl; —O(C₁-C₆ haloalkoxy) C₁-C₆ alkyl; aryloxy, sulfhydryl (mercapto), halo (C₁-C₆) alkyl (e.g., —CF₃), halo (C₁-C₆) alkoxy (e.g., —OCF₃), C₁-C₆ alkylthio, arylthio, amino, amino (C₁-C₆) alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁-4alkyl, monophosphate, diphosphate, triphosphate, and oxo (═O). Wherever a group is described as “optionally substituted” that group can be substituted with the above substituents.

As understood by one of ordinary skill in the art, a compound described herein may exist in ionized form, e.g., —CO₂, —SO₃ or —O—SO₃. If a compound contains a positively or negatively charged substituent group, for example, —SO₃, it may also contain a negatively or positively charged counterion such that the compound as a whole is neutral. In other aspects, the compound may exist in a salt form, where the counterion is provided by a conjugate acid or base.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxy group that is present in ribose. The nitrogen containing heterocyclic base can be purine or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, deazapurine, 7-deaza adenine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” or “incorporating” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative,” “analog,” or “modified” means a compound or molecule whose core structure is the same as, or closely resembles that of a parent compound but which has a chemical or physical modification, such as, for example, a different or additional side group. “Derivative,” “analog,” and “modified” as used herein, may be used interchangeably. When referring to nucleotides or nucleosides, “derivative,” “analog,” or “modified” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative,” “analog,” and “modified” can be encompassed by the terms “nucleotide” and “nucleoside” defined herein.

As used herein, the term “phosphate” is used in its ordinary sense as understood by those skilled in the art, and includes its protonated forms (for example,

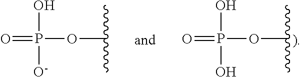 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Pre-phasing is caused by the incorporation of nucleotides without effective 3′ terminators, wherein the incorporation event goes 1 cycle ahead due to a termination failure. Phasing and pre-phasing cause the measured signal intensities for a specific cycle to consist of the signal from the current cycle as well as noise from the preceding and following cycles. As the number of cycles increases, the fraction of sequences per cluster affected by phasing and pre-phasing increases, hampering the identification of the correct base. Pre-phasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes. Pre-phasing can be caused by residual cleave mixture on the flowcell due to insufficient washing after introduction of the cleave mixture. Accordingly, the discovery of nucleotide analogues which decrease the incidence of pre-phasing is surprising and provides a great advantage in SBS applications over existing nucleotide analogues. For example, the nucleotide analogues provided can result in faster SBS cycle time, lower phasing and pre-phasing values, and longer sequencing read lengths.

As used herein, the term “macrocycle” refers to molecules and ions containing a ring of twelve or more atoms. Macrocycles may include but are not limited to cyclodextrins, calixarenes, cucurbiturils, crown ethers, and porphyrins.

### Sequencing Methods Using Scan Mixtures Containing Macrocycle Additives

One aspect of the present disclosure relates to a method of sequencing a plurality of different target polynucleotides, comprising:
(a) contacting a solid support with an incorporation mixture comprising DNA polymerase and one or more of four different types of nucleotides (e.g., dATP, dCTP, dGTP, and dTTP or dUTP), wherein the solid support comprises a plurality of different target polynucleotides immobilized thereon, and sequencing primers that are complementary and hybridized to at least a portion of the target polynucleotides;
(b) incorporating one type of nucleotides into the sequencing primers to produce extended copy polynucleotides, wherein one or more of the four types of nucleotides comprises a detectable label; and
(c) imaging and performing one or more fluorescent measurements of the extended copy polynucleotides in an aqueous scan mixture;

wherein the aqueous scan mixture comprises one or more additives for enhancing fluorescent signal intensity of the detectable label, and wherein the one or more additives comprise one or more water-soluble macrocycles.

In some embodiments of the method described herein, the water-soluble macrocycle forms a host-guest complex with the detectable label, as such the macrocycle encapsulates the detectable label. In some embodiments, the water-soluble macrocycle comprises water-soluble cyclodextrins, water-soluble calixarenes, water-soluble cucurbiturils, or optionally substituted analogs, salts (e.g., an acid addition salt such as HCl salt, or an alkali metal salt such as KCl or NaCl), or hydrates thereof, or combination thereof.

In some embodiments of the method described herein, the water-soluble cyclodextrins or the optionally substituted analogs, salts, or hydrates thereof comprise α-cyclodextrin, β-cyclodextrin, γ-cyclodextrin, or substituted analogs or salts thereof, or combination thereof. In some embodiments, the substituted analogs of the water-soluble cyclodextrins are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the water-soluble cyclodextrin or the substituted analogs, salts, or hydrates thereof are selected from the group consisting of α-cyclodextrin, β-cyclodextrin, (2-hydroxypropl)-β-cyclodextrin, acetyl-β-cyclodextrin, (2-hydroxyethyl)-β-cyclodextrin, heptakis (2,3,6-tri-O-methyl)-β-cyclodextrin, succinyl-β-cyclodextrin, methyl-β-cyclodextrin, carboxymethyl-β-cyclodextrin, heptakis (6-deoxy-6-amino)-β-cyclodextrin heptahydrochloride, γ-cyclodextrin hydrate, (2-hydroxypropyl)-γ-cyclodextrin, and salts and combinations thereof. In further embodiments, the water-soluble cyclodextrin comprises or is (2-hydroxyporpyl)-β-cyclodextrin. In further embodiments, the water-soluble cyclodextrin comprises or is methyl-β-cyclodextrin. In some embodiments, the binding affinity constant (Kb) of the water-soluble cyclodextrin to the detectable label is at least about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,500, 3,000, 3,500, 4,000, 4,500, or 5,000 M-1, or in a range defined by any two of the preceding values. In certain embodiments, the binding affinity constant of the water-soluble cyclodextrin to the detectable label is at least about 100, 200, 300, 400, or 500 M-1. In some embodiments, the water-soluble cyclodextrin or the optionally substituted analog, salt or hydrate thereof in the scan mixture increases fluorescence intensity by at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%, or a range defined by any two of the preceding values as compared to the fluorescence intensity of the same sequencing runs using a control scan mixture without the addition of the water-soluble cyclodextrin in any read cycle (e.g., at cycle 26, 50, 75, 100, 125, 150, 200, 250, 300, 350, 400, 450, or 500). In some embodiments, the fluorescence intensity is green fluorescence intensity, referring to the emission from an imaging event from a green light source (e.g., a light source having a wavelength from about 520 nm to about 560 nm). In some other embodiments, the fluorescence intensity is blue fluorescence intensity, referring to the emission from an imaging event from a blue light source (e.g., a light source having a wavelength from about 450 nm to about 460 nm). In certain embodiments, the water-soluble cyclodextrin or the analog, salt or hydrate thereof in the scan mixture increases blue fluorescence intensity by at least about 10%, 15%, 20%, 25%, 30%, 35%,40%, 45%, 50%, 55%, 60%, 65%, 70%, 75%, 80%, 85%, 90%, 95%, or 100% in read cycle 26. In certain embodiments, the water-soluble cyclodextrin increases green fluorescence intensity by at least about 10%, 15%, 20%, 25%, 30%, 35%, 40%, 45%, 50%, 55%, 60%, 65%, 70%, 75%, 80%, 85%, 90%, 95%, or 100% in read cycle 26. In some embodiments, the water-soluble cyclodextrin reduces photo bleaching and/or harmful radical generated during sequencing (e.g., the singlet oxygen production) resulted from the imaging event during sequence.

In some embodiments of the method described herein, the water-soluble cucurbituril or the optionally substituted analogs, salts, or hydrates thereof comprise cucurbituril hydrates or substituted analogs or salts thereof, or combination thereof. In some embodiments, the cucurbituril hydrates comprise or are selected from the group consisting of cucurbit [5]uril hydrate, cucurbit [6]uril hydrate, cucurbit [7]uril hydrate, and cucurbit [8]uril hydrate. In some further embodiments, the cucurbituril hydrate comprises or is cucurbit [7]uril hydrate. In some embodiments, the substituted analogs of the water-soluble cucurbiturils are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the binding affinity constant of the water-soluble cucurbituril to the detectable label is at least about 1,000, 10,000, 25,000, 50,000, 75,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, or 1,000,000 M 1 or in a range defined by any two of the preceding values. In some embodiments, the binding affinity constant of the water-soluble cucurbituril to the detectable label is at least about 1×10⁴M⁻¹, 2×10⁴M⁻¹, 3×10⁴M⁻¹, 4×10⁴M⁻¹, 5×10⁴M⁻¹, 6×10⁴M⁻¹, 7×10⁴M⁻¹, 8×10⁴M⁻¹ 9×10⁴M⁻¹, 1×10⁵M⁻¹, 2×10⁵M⁻¹, or 3×10⁵M⁻¹. In some further embodiments, the binding affinity constant of the water-soluble cucurbituril to the detectable label is at least about 1×10⁵M⁻¹.

In some embodiments of the method described herein, the water-soluble calixarenes or the optionally substituted analogs, salts, or hydrates thereof comprise sulfocalixarenes or substituted analogs or salts thereof, or combination thereof. In some embodiments, the substituted analogs of the water-soluble calixarenes are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the water-soluble calixarenes or the substituted analogs, salts, or hydrates thereof comprise or are selected from the group consisting of 4-sulfocalix [4] arene, 4-sulfocalix [4] arene hydrate, 4-sulfothiacalix [4] arene, and salts and combinations thereof. In some embodiments, the binding affinity constant of the water-soluble calixarene to the detectable label is at least about 500, 1000, 1500, 2000, 2500, 3000, 3500, 4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19000, 20,000 or 25,000, or in a range defined by any two of the preceding values.

In some embodiments of the method described herein, the one or more water-soluble macrocycles also reduces the singlet oxygen production, as compared to the same sequencing run(s) where the scan mixture does not have the one or more water-soluble macrocycles described herein. In some embodiments, the one or more water-soluble macrocycles reduce singlet oxygen production by at least 5%, 10%, 15%, 20%, 25%, 30%, 35%, 40%, 45%, 50%, 55%, 60%, 65%, 70%, 75%, 80%, 85%, 90%, 95%, or 100% or in a range defined by any two of the preceding values.

In any embodiments of the method described herein, the detectable label is a fluorescent dye. In some embodiments, the fluorescent dye is excitable by a light source having a wavelength between about 400 nm to 600 nm, from about 450 nm to about 550 nm, from about 450 nm to about 460 nm or from about 520 nm to about 540 nm. In some embodiments, the fluorescent dye is a blue dye. In further embodiments, the blue dye may be a coumarin or a coumarin derivative. In some other embodiments, the dye moiety is a green dye.

### Macrocycles

In some embodiments, macrocycle additives to scan mixture are water soluble. In some embodiments of the method described herein, the water-soluble macrocycle forms a host-guest complex with the detectable label. In some embodiments, the water-soluble macrocycle comprises water-soluble cyclodextrins, water-soluble calixarenes, water-soluble cucurbiturils, or optionally substituted analogs, salts (e.g., an acid addition salt such as HCl salt, or an alkali metal salt such as KCl or NaCl), or hydrates thereof, or combination thereof.

In some embodiments of the method described herein, the water-soluble cyclodextrins or the optionally substituted analogs, salts, or hydrates thereof comprise α-cyclodextrin, β-cyclodextrin, γ-cyclodextrin, or substituted analogs or salts thereof, or combination thereof. In some embodiments, the substituted analogs of the water-soluble cyclodextrins are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the water-soluble cyclodextrin or the substituted analogs, salts, or hydrates thereof comprise or are selected from the group consisting of α-cyclodextrin, β-cyclodextrin, (2-hydroxypropl)-β-cyclodextrin, acetyl-β-cyclodextrin, (2-hydroxyethyl)-β-cyclodextrin, heptakis (2,3,6-tri-O-methyl)-β-cyclodextrin, succinyl-β-cyclodextrin, methyl-β-cyclodextrin, carboxymethyl-β-cyclodextrin, heptakis (6-deoxy-6-amino)-β-cyclodextrin heptahydrochloride, γ-cyclodextrin hydrate, (2-hydroxypropyl)-γ-cyclodextrin, and salts and combinations thereof. In further embodiments, the water-soluble cyclodextrin comprises or is (2-hydroxyporpyl)-cyclodextrin. In some embodiments, the concentration of water-soluble cyclodextrin or the optionally substituted analogs, salts, or hydrates thereof in the scan mixture is about 0.1, 0.5, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750 775, 800, 825, 850, 875, 900, 925, 950, 975, 1,000, 2,000, 3,000, 4,000, 5,000 mM or in a range defined by any two of the preceding values. In some further embodiments, the concentration of water-soluble cyclodextrin or the optionally substituted analog, salt, or hydrate thereof in the scan mixture is about 0.1 mM to about 500 mM, about 0.2 mM to about 400 mM, about 0.4 mM to about 200 mM, about 0.6 mM to about 100 mM, about 0.8 mM to about 50 mM, about 1 mM to about 10 mM, about 2 mM to about 8 mM, about 4 mM to about 6 mM, or about 5 mM. In some further embodiments, the concentration of the water-soluble cyclodextrin or the optionally substituted analog, salt, or hydrate thereof in the aqueous scan mixture is from about 1 mM to about 500 mM, about 5 mM to about 400 mM, about 10 mM to about 300 mM, or about 20 mM to about 150 mM.

In some embodiments, the water-soluble cucurbituril or the optionally substituted analogs, salts, or hydrates thereof comprise cucurbituril hydrates or substituted analogs or salts thereof, or combination thereof. In some embodiments, the cucurbituril hydrates comprise or are selected from the group consisting of cucurbit [5]uril hydrate, cucurbit [6]uril hydrate, cucurbit [7]uril hydrate, and cucurbit [8]uril hydrate. In some further embodiments, the cucurbituril hydrate comprises or is cucurbit [7]uril hydrate. In some embodiments, the substituted analogs of the water-soluble cucurbiturils are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the concentration of water-soluble cucurbituril or the optionally substituted analog, salt, or hydrate thereof is about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, or 50 mM, or in a range defined by any two of the preceding values. In some further embodiments, the concentration of water-soluble cucurbituril or the optionally substituted analog, salt, or hydrate thereof in the scan mixture is about 0.001 mM to about 1 mM, about 0.005 mM to about 0.5 mM, about 0.01 mM to about 0.1 mM, about 0.02 mM to about 0.08 mM, about 0.04 mM to about 0.06 mM, or about 0.05 mM.

In some embodiments, the water-soluble calixarenes or the optionally substituted analogs, salts, or hydrates thereof comprise sulfocalixarenes or substituted analogs or salts thereof, or combination thereof. In some embodiments, the substituted analogs of the water-soluble calixarenes are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof. In some embodiments, the water-soluble calixarenes or the substituted analogs, salts, or hydrates thereof are selected from the group consisting of 4-sulfocalix [4] arene, 4-sulfocalix [4] arene hydrate, 4-sulfothiacalix [4] arene, and salts and combinations thereof. In some embodiments, the concentration of water-soluble calixarene, or the optionally substituted analog, salt, or hydrate thereof in the scan mixture is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, or 500 mM or in a range defined by any two of the preceding values. In some further embodiments, the concentration of water-soluble calixarene or the optionally substituted analog, salt, or hydrate thereof in the scan mixture is about 0.01 mM to about 10 mM, about 0.05 mM to about 5 mM, about 0.1 mM to about 1 mM, about 0.2 mM to about 0.8 mM, about 0.4 mM to about 0.6 mM, or about 0.5 mM.

The addition of macrocycles to a scan mixture can enhance sequencing performance. For example, macrocycles may facilitate a reduced laser power requirement, which in turn may allow for longer reads and/or improve data quality. In some embodiments, inclusion of macrocycles may allow for reduced light exposure per SBS cycle but comparable signal as is currently achieved in SBS. Such reduced light exposure may reduce photo bleaching, signal decay, and/or error rates. In some embodiments, inclusion of macrocycles in scan mixture paired with the same and/or similar light exposure as currently used in SBS may allow for increased scanning speed, which may further allow for faster cycles of SBS. In some embodiments, inclusion of macrocycles in scan mixture with light exposure that may be ramped and/or changed through an SBS run may thereby allow longer reads and improved percentage of Q30 score for longer cycles.

Macrocycles described herein may be used to preserve and/or enhance various dyes. Dyes may include fluorescent dyes used as detectable labels, in particularly those dyes that are excitable by a blue light (e.g., about 450 nm to about 460 nm, or about 450 nm to about 495 nm) or a green light (e.g., about 520 nm to about 540 nm, or about 495 nm to about 570 nm). Dyes may also include fluorescent dyes that are excitable by a cyan light (e.g., about 490 nm to about 520 nm). These dyes may also be referred to as “blue dyes,” “green dyes,” and “cyan dyes” respectively, referring to the wavelength/color of the excitation light source(s). Examples of various type of blue dyes, including but not limited to coumarin dyes, chromenoquinoline dyes, and bisboron containing heterocycles are disclosed in U.S. Publication Nos. 2018/0094140, 2018/0201981, 2020/0277529, 2020/0277670, 2021/0188832 and 2022/0033900, and U.S. Ser. Nos. 17/550,271, 17/736,688, and 63/325,057, each of which is incorporated by reference in its entirety. Examples of green dyes including cyanine or polymethine dyes disclosed in International Publication Nos. WO2013/041117, WO2014/135221, WO 2016/189287, WO2017/051201 and WO2018/060482A1, each of which is incorporated by reference in its entirety.

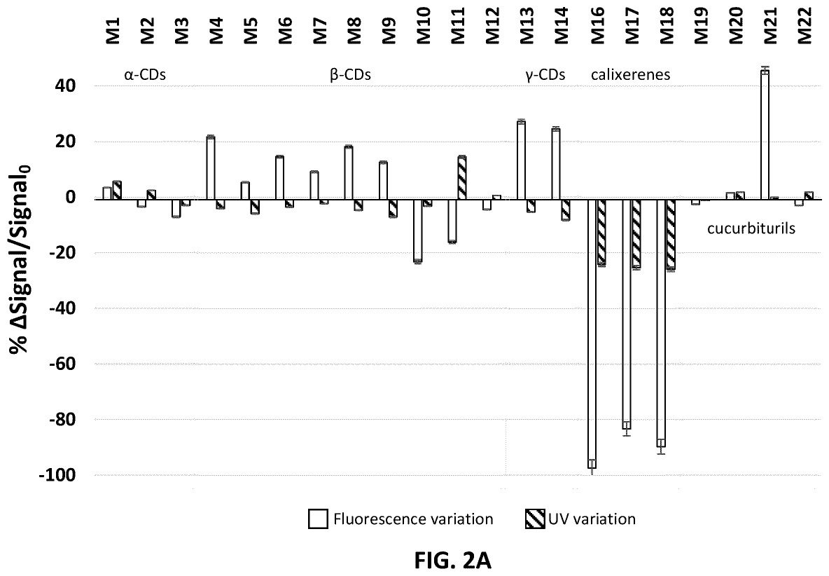

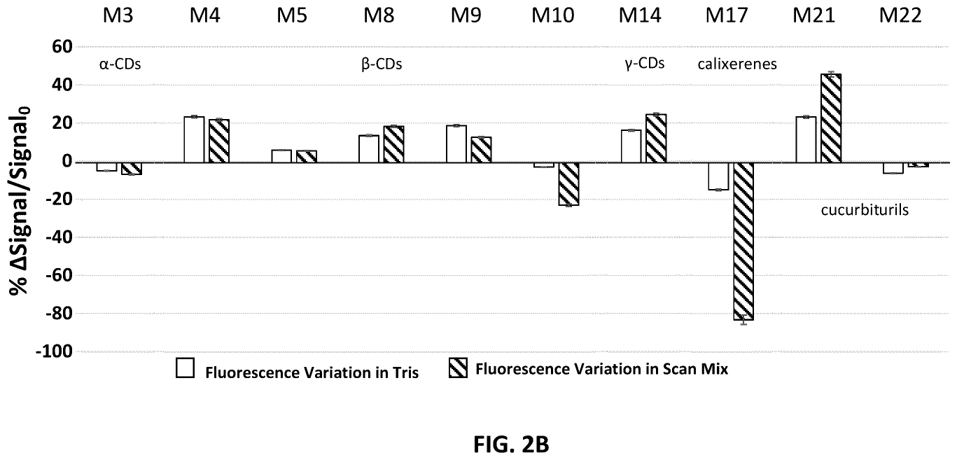

Table 1 lists several example macrocycles and their structures. Additionally, Table 1 lists the macrocycles' designators, used as abbreviations in FIGS. 2A-6B.

TABLE 1
Exemplary Macrocycles

| | | |
|---|---|---|
| Molecule Name | Designator | Structure |

α-Cyclodextrins

| | | |
|---|---|---|
| α-Cyclodextrin | M1 | 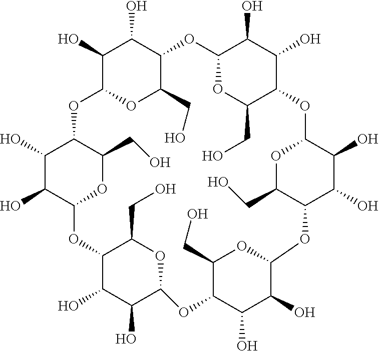 [SMILES: OC[C@H]1O[C@@H]2O[C@H]3[C@@H](O)[C@H](O)[C@@H](O[C@H]4[C@@H](O)[C@H](O)[C@@H](O[C@H]5[C@@H](O)[C@H](O)[C@@H](O[C@H]6[C@@H](O)[C@H](O)[C@@H](O[C@H]7[C@@H](O)[C@H](O)[C@@H](O[C@H]1[C@@H](O)[C@@H]2O)O[C@@H]7CO)O[C@@H]6CO)O[C@@H]5CO)O[C@@H]4CO)O[C@@H]3CO] |
| α-Cyclodextrin, sulfated sodium salt hydrate | M2 | 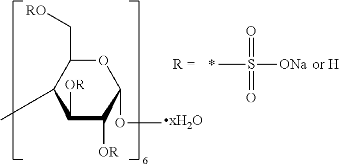 |

β-Cyclodextrins

| | | |
|---|---|---|
| β-Cyclodextrin | M3 | 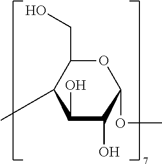 |
| (2-Hydroxypropyl)-β- cyclodextrin | M4 | 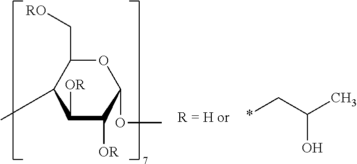 |
| Acetyl-β-cyclodextrin | M5 | 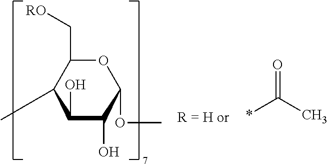 |
| (2-Hydroxyethyl)-β- cyclodextrin | M6 | 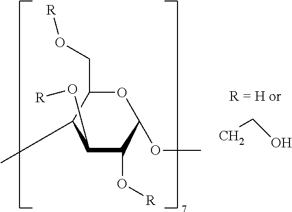 |
| Heptakis(2,3,6-tri-O- methyl)-β-cyclodextrin | M7 | 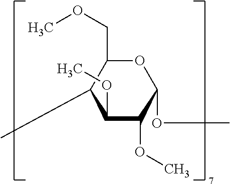 |
| Succinyl-β-cyclodextrin | M8 | |
| Methyl-β-cyclodextrin | M9 | 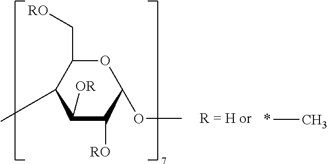 |
| Carboxymethyl-β- cyclodextrin sodium salt | M10 | 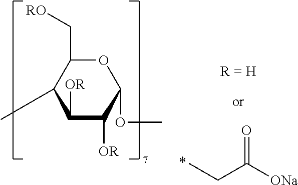 |
| Heptakis(6-deoxy-6- amino)-β-cyclodextrin Heptahydrochloride | M11 | 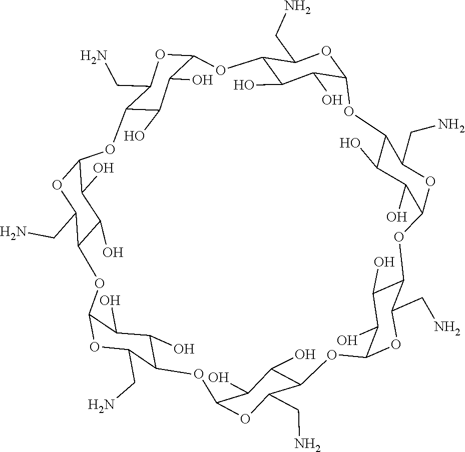 [SMILES: NCC1OC2OC3C(CN)OC(OC4C(CN)OC(OC5C(CN)OC(OC6C(CN)OC(OC7C(CN)OC(OC8C(CN)OC(OC1C(O)C2O)C(O)C8O)C(O)C7O)C(O)C6O)C(O)C5O)C(O)C4O)C(O)C3O] |
| β-Cyclodextrin SULFATED SODIUM | M12 | 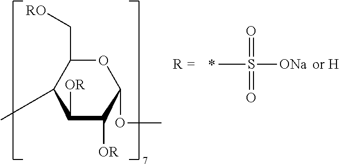 |

γ-Cyclodextrins

| | | |
|---|---|---|
| γ-Cyclodextrin hydrate | M13 | 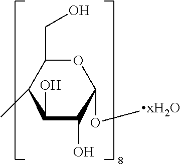 |
| (2-Hydroxypropyl)-γ- cyclodextrin | M14 | 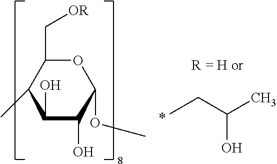 |

Calixarenes

| | | |
|---|---|---|
| 4-Sulfocalix[4]arene | M16 | 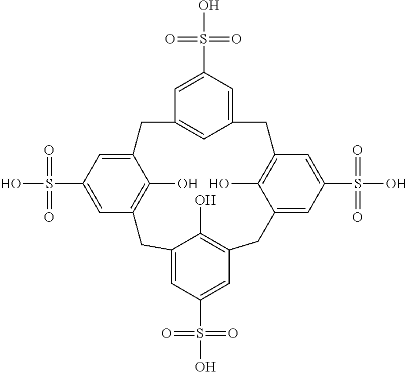 [SMILES: O=S(=O)(O)c1cc2cc(c1)Cc1cc(S(=O)(=O)O)cc(c1O)Cc1cc(S(=O)(=O)O)cc(c1O)Cc1cc(S(=O)(=O)O)cc(c1O)C2] |
| 4-Sulfocalix[6]arene Hydrate | M17 | 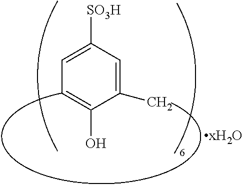 |
| 4-Sulfothiacalix[4]arene Sodium Salt | M18 | 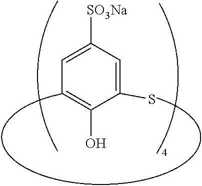 |

Cucurbiturils

| | | |
|---|---|---|
| Cucurbit[5]uril hydrate | M19 | 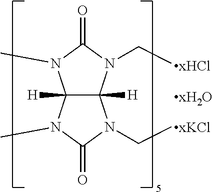 |
| Cucurbit[6]uril hydrate | M20 | 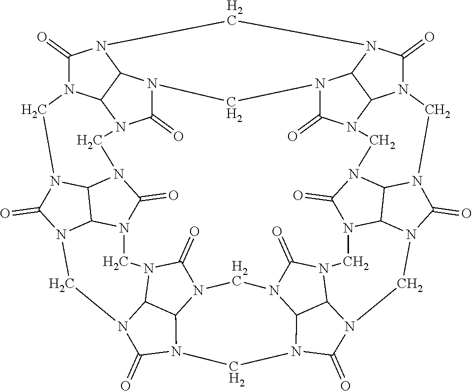 [SMILES: O=C1N2CN3C(=O)N4CN5C(=O)N6CN7C(=O)N8CN9C(=O)N%10CN%11C(=O)N%12CN1C1C2N2CN%13C(=O)N(CN%14C(=O)N(CN%15C(=O)N(CN%16C(=O)N(CN%17C(=O)N(CN1C2=O)C%12C%11%17)C%10C9%16)C8C7%15)C6C5%14)C4C3%13] |
| Cucurbit[7]uril hydrate | M21 | 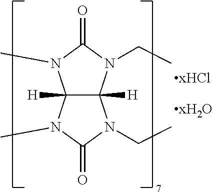 |
| Cucurbit[8]uril hydrate | M22 | 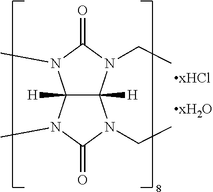 |

Nucleotides with 3′ Blocking Groups

Some embodiments of the present disclosure relate to a nucleotide molecule comprising a nucleobase, a ribose or deoxyribose moiety, and a 3′ hydroxy blocking group comprising an unsubstituted or substituted allyl moiety, such as a 3′ blocking group having the structure

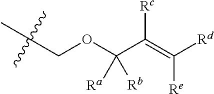

attached to the 3′ oxygen of the nucleotide, wherein each of R^{a}, R^{b}, R^{c}, R^{d} and R^{e} is independently H, halogen, unsubstituted or substituted C₁-C₆ alkyl, or C₁-C₆ haloalkyl. In one embodiment, each of R^{a}, R^{b}, R^{c}, R^{d} and R^{e} is H. In some other embodiments, each of R^{a} and R^{b} is H and at least one of R^{e}, R^{d} and R^{e} is independently halogen (e.g., fluoro, chloro) or unsubstituted C₁-C₆ alkyl (e.g., methyl, ethyl, isopropyl, isobutyl, or t-butyl). For example, R^{e} is unsubstituted C₁-C₆ alkyl and each of R^{d} and R^{e} is H. In another example, R^{e} is H and one or both of R^{d} and R^{e} is halogen or unsubstituted C₁-C₆ alkyl. Non-limiting embodiments of the 3′ blocking group include

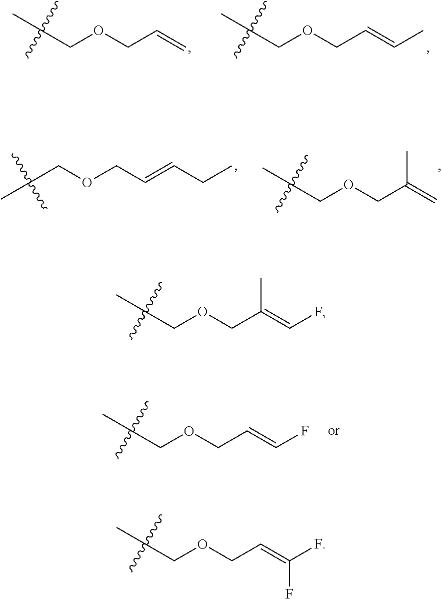

In one embodiment, the 3′ blocking group is

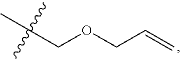 [SMILES: *COCC=C]

and together with the 3′ oxygen it forms

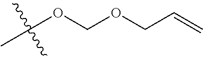 [SMILES: *OCOCC=C]

(“AOM”) group attached to the 3′ carbon atom of the ribose or deoxyribose moiety. Additional embodiments of the 3′ blocking groups are described in U.S. Publication No. 2020/0216891 A1, which is incorporated by reference in its entirety. In any embodiments of the nucleotide described herein, the nucleotide may comprise a 3′ blocked 2-deoxyribose moiety. Furthermore, the nucleotide may be a nucleoside triphosphate. In another embodiment, the 3′ blocking group is an allyl ether group (—O—CH₂CH═CH₂), attached to the 3′ carbon atom of the deoxyribose moiety.

### Deprotection of the 3′ Blocking Groups

In some embodiments, the 3′ blocking group may be removed or deprotected by a chemical reagent to generate a free hydroxy group, for example, in the presence of a water soluble phosphine reagent. Non-limiting examples include tris (hydroxymethyl) phosphine (THMP), tris (hydroxyethyl) phosphine (THEP) or tris (hydroxylpropyl) phosphine (THP or THPP). 3′-acetal blocking groups described herein may be removed or cleaved under various chemical conditions. For 3′ acetal blocking groups such as

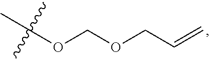 [SMILES: *OCOCC=C]

non-limiting cleaving condition includes a Pd(II) complex, such as Pd(OAc) 2 or allylPd (II) chloride dimer, in the presence of a phosphine ligand, for example tris (hydroxymethyl) phosphine (THMP), or tris (hydroxylpropyl) phosphine (THP or THPP). For those blocking groups containing an alkynyl group (e.g., an ethynyl), they may also be removed by a Pd(II) complex (e.g., Pd(OAc) 2 or allyl Pd(II) chloride dimer) in the presence of a phosphine ligand (e.g., THP or THMP).

### Palladium Cleavage Reagents

In some other embodiments, the 3′ blocking group described herein such as allyl or AOM may be cleaved by a palladium catalyst. In some such embodiments, the Pd catalyst is water soluble. In some such embodiments, is a Pd (0) complex (e.g., Tris (3,3′,3″-phosphinidynetris (benzenesulfonato) palladium (0) nonasodium salt nonahydrate). In some instances, the Pd (0) complex may be generated in situ from reduction of a Pd(II) complex by reagents such as alkenes, alcohols, amines, phosphines, or metal hydrides. Suitable palladium sources include Na₂PdCl₄, Li₂PdCl₄, Pd(CH₃CN)₂Cl₂. (PdCI(C₃H₅))₂, [Pd(C₃H₅) (THP)]Cl, [Pd(C₃H₅)(THP)₂]Cl, Pd(OAc)₂, Pd(Ph₃)₄, Pd(dba)₂, Pd(Acac)₂, PdCl₂(COD), Pd(TFA)₂, Na₂PdBr₄, K₂PdBr₄, PdCl₂, PdBr₂, and Pd(NO₃)₂. In one such embodiment, the Pd (0) complex is generated in situ from Na₂PdCl₄ or K₂PdCl₄. In another embodiment, the palladium source is allyl palladium (II) chloride dimer [(PdCI(C₃H₅)) 2]. In some embodiments, the Pd (0) complex is generated in an aqueous solution by mixing a Pd(II) complex with a phosphine. Suitable phosphines include water soluble phosphines, such as THP, THMP, PTA, TCEP, bis (p-sulfonatophenyl) phenylphosphine dihydrate potassium salt, or triphenylphosphine-3,3′,3″-trisulfonic acid trisodium salt.

In some embodiments, the palladium catalyst is prepared by mixing [(Allyl) PdCI]2 with THP in situ. The molar ratio of [(Allyl) PdCl]2 and the THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of [(Allyl) PdCl]2 to THP is 1:10. In some other embodiment, the palladium catalyst is prepared by mixing a water soluble Pd reagent such as Na₂PdCl₄ or K₂PdCl₄ with THP in situ. The molar ratio of Na₂PdCl₄ or K₂PdCl₄ and THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5. In yet another embodiment, the molar ratio of Na₂PdCl₄ or K₂PdCl₄ to THP is about 1:2.5. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris (hydroxymethyl) aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine (TEMED), N,N,N′,N′-tetraethylethylenediamine (TEEDA), or 2-piperidine ethanol (also known as (2-hydroxyethyl) piperidine, having the structure

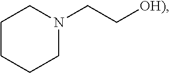

or combinations thereof. In one embodiment, the buffer reagent comprises or is DEEA. In another embodiment, the buffer reagent comprises or is (2-hydroxyethyl) piperidine. In another embodiment, the buffer reagent contains one or more inorganic salts such as a carbonate salt, a phosphate salt, or a borate salt, or combinations thereof. In one embodiment, the inorganic salt is a sodium salt.

### Cleavable Linkers

The dye compounds as disclosed herein may include a reactive linker group at one of the substituent positions for covalent attachment of the compound to a substrate or another molecule. Reactive linking groups are moieties capable of forming a bond (e.g., a covalent or non-covalent bond), in particular a covalent bond. In a particular embodiment the linker may be a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO 2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO 2004/018493 (herein incorporated by reference) such as those that include moieties of the formulae:

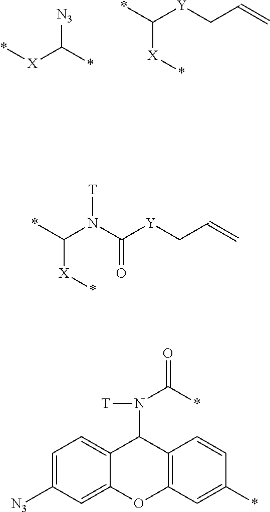

(wherein X is selected from the group comprising O, S, NH and NQ wherein Q is a C₁-10 substituted or unsubstituted alkyl group, Y is selected from the group comprising O, S, NH and N (allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

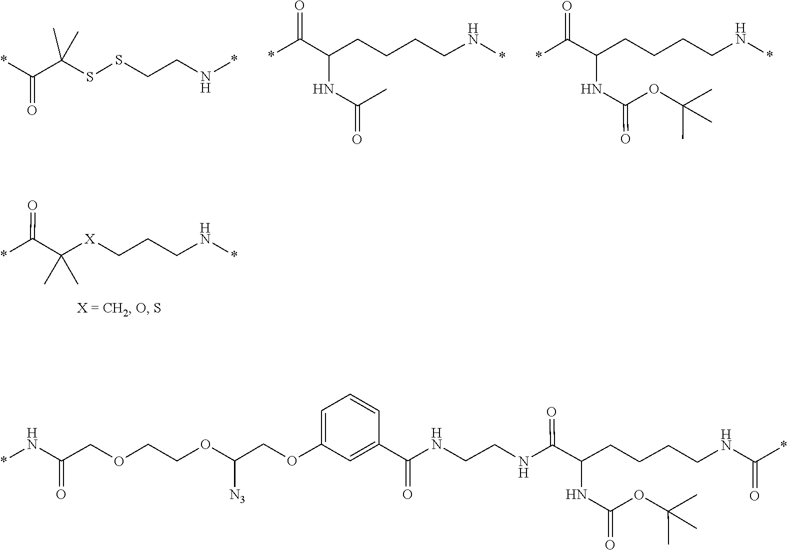

(wherein * indicates where the moiety is connected to the remainder of the nucleotide or nucleoside). The linker moieties illustrated herein may comprise the whole or partial linker structure between the nucleotides/nucleosides and the labels. The linker moieties illustrated herein may comprise the whole or partial linker structure between the nucleotides/nucleosides and the labels.

Additional examples of linkers include moieties of the formula:

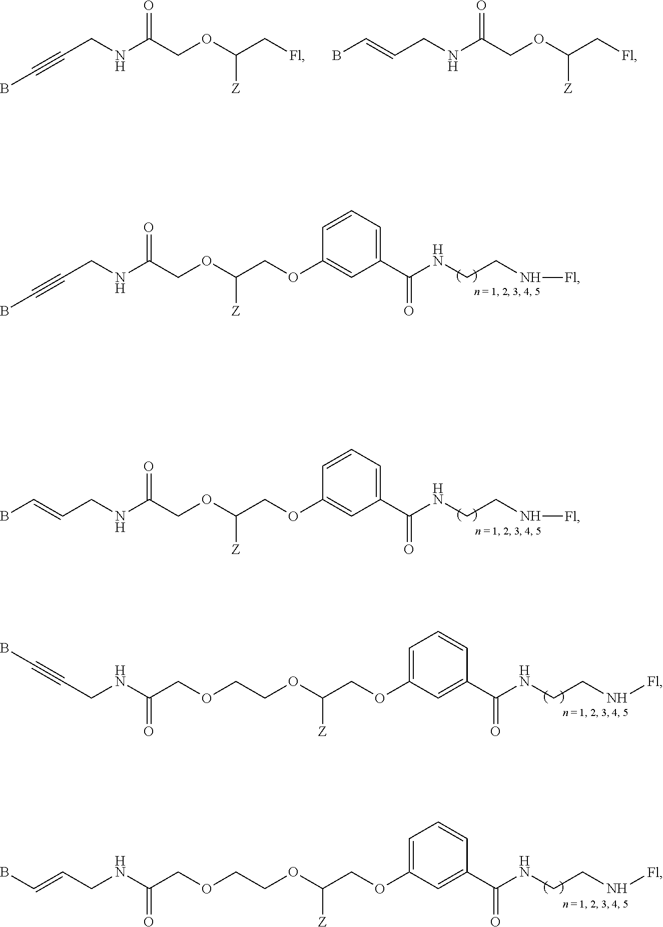

wherein B is a nucleobase; Z is —N₃ (azido), —O—C₁-C₆ alkyl, —O—C₂-C₆ alkenyl, or —O—C₂-C₆ alkynyl; and F1 comprises a dye moiety, which may contain additional linker structure. One of ordinary skill in the art understands that the dye compound described herein is covalently bounded to the linker by reacting a functional group of the dye compound (e.g., carboxyl) with a functional group of the linker (e.g., amino). In one embodiment, the cleavable linker comprises

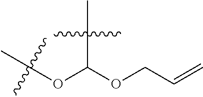 [SMILES: *OC(*)OCC=C]

(“AOL” linker moiety) where Z is-O-allyl.

In particular embodiments, the length of the linker between a fluorescent dye (fluorophore) and a guanine base can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO 2007/020457 (herein incorporated by reference). The design of linkers, and especially their increased length, can allow improvements in the brightness of fluorophores attached to the guanine bases of guanosine nucleotides when incorporated into polynucleotides such as DNA. Thus, when the dye is for use in any method of analysis which requires detection of a fluorescent dye label attached to a guanine-containing nucleotide, it is advantageous if the linker comprises a spacer group of formula —((CH₂)₂O)ₙ—, wherein n is an integer between 2 and 50, as described in WO 2007/020457.

Nucleosides and nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono, di- or triphosphates.

A “nucleoside” is structurally similar to a nucleotide but is missing the phosphate moieties. An example of a nucleoside analog would be one in which the label is linked to the base and there is no phosphate group attached to the sugar molecule.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular embodiments, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidate linkages and the like.

A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleoside or nucleotide.

In particular embodiments the labeled nucleotide or oligonucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

### Labeled Nucleotides

In some embodiments, the 3′ blocked nucleotide also comprises a detectable label and such nucleotide is called a labeled nucleotide or a fully functionalized nucleotide (ffN). The label (e.g., a fluorescent dye) is conjugated via a cleavable linker by a variety of means including hydrophobic attraction, ionic attraction, and covalent attachment. In some aspect, the dyes are conjugated to the nucleotide by covalent attachment via the cleavable linker. One of ordinary skill in the art understands that a label may be covalently bounded to the linker by reacting a functional group of the label (e.g., carboxyl) with a functional group of the linker (e.g., amino). The label may be, for example, a dye. In some such embodiments, the cleavable linker may comprise a moiety that is the same as the 3′ blocking group. As such, the cleavable linker and the 3′ blocking group may be cleaved or removed under the same reaction condition. In some such embodiments, the cleavable linker may comprise an allyl moiety, more particularly comprises a moiety of the structure:

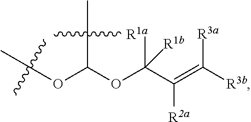

wherein each of R^{1a}, R^{1b}, R^{2a}, R^{3a} and R^{3b} is independently H, halogen, unsubstituted or substituted C₁-C₆ alkyl, or C₁-C₆ haloalkyl.

In some embodiments, the dye may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a cleavable linker moiety.

Nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine (e.g., deazapurine, 7-deazapurine) or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono, di- or triphosphates.

Although the base is usually referred to as a purine or pyrimidine, the skilled person will appreciate that derivatives and analogues are available which do not alter the capability of the nucleotide or nucleoside to undergo Watson-Crick base pairing. In the context of nucleotide derivatives and/or nucleoside derivatives, such modifications may allow the derivative nucleotide or nucleoside to be linked to another molecule. For example, the base may be a deazapurine. In particular embodiments, the derivatives should be capable of undergoing Watson-Crick pairing. In the context of nucleotides and nucleosides, “derivative” and “analog” also include, for example, a synthetic nucleotide or nucleoside derivative having modified base moieties and/or modified sugar moieties. Such derivatives and analogs are discussed in, for example, Scheit, Nucleotide analogs (John Wiley & Son, 1980) and Uhlman et al., Chemical Reviews 90:543-584, 1990. Nucleotide analogs can also comprise modified phosphodiester linkages including phosphorothioate, phosphorodithioate, alkyl-phosphonate, phosphoranilidate, phosphoramidite linkages and the like.

In particular embodiments, the labeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

The disclosure also encompasses polynucleotides incorporating a nucleotide described herein. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

In some embodiments, the labeled nucleotide described herein comprises or has the structure of Formula (I):

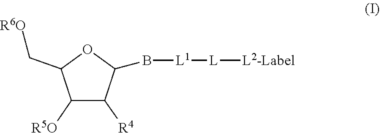

wherein B is the nucleobase;
R⁴ is H or OH;
R⁵ is an allyl containing 3′ blocking group, such as

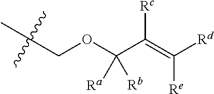

as described herein or —OR⁵ is a phosphoramidite;
R⁶ is H, monophosphate, diphosphate, triphosphate, thiophosphate, a phosphate ester analog, a reactive phosphorous containing group, or a hydroxy protecting group;
L is an allyl moiety containing linker, such as

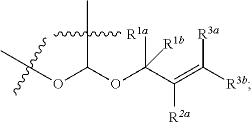

and each of L¹ and L² is independently an optionally present linker moiety.

In some embodiments of the nucleotide described herein, each of R^{1a}, R^{1b}, R^{2a}, R^{3a} and R^{3b} is H. In other embodiments, at least one of R^{1a}, R^{1b}, R^{2a}, R^{3a} and R^{3b} is halogen (e.g., fluoro, chloro) or unsubstituted C₁-C₆ alkyl (e.g., methyl, ethyl, isopropyl, isobutyl, or t-butyl). In some such instances, each of R^{1a} and R^{1b} is H and at least one of R^{2a}, R^{3a} and R^{3b} is unsubstituted C₁-C₆ alkyl or halogen (for example, R^{2a} is unsubstituted C₁-C₆ alkyl and each of R^{3a} and R^{3b} is H; or R^{2a} is H and one or both of R^{3a} and R^{3b} is halogen or unsubstituted C₁-C₆ alkyl). In one embodiment, the cleavable linker or L comprises

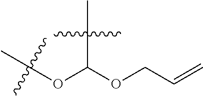 [SMILES: *OC(*)OCC=C]

(“AOL” linker moiety).

In some embodiments of the nucleotide described herein, the nucleobase (“B” in Formula (I)) is purine (adenine or guanine), a deaza purine, or a pyrimidine (e.g., cytosine, thymine or uracil). In some further embodiments, the deaza purine is 7-deaza purine (e.g., 7-deaza adenine or 7-deaza guanine). Non-limiting examples of B comprises

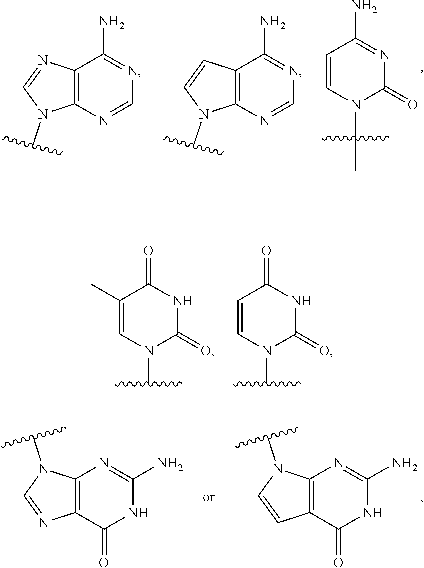

or optionally substituted derivatives and analogs thereof. In some further embodiments, the labeled nucleobase comprises the structure

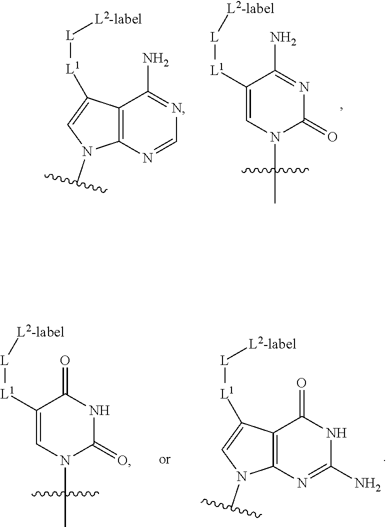

In some other embodiments of the nucleotide described herein, R⁵ in Formula (I) is a phosphoramidite. In such embodiments, R⁶ is an acid-cleavable hydroxy protecting group which allows subsequent monomer coupling under automated synthesis conditions.

In some embodiments of the nucleotide described herein, L¹ is present and L¹ comprises a moiety selected from the group consisting of a propargylamine, a propargylamide, an allylamine, an allylamide, and optionally substituted variants thereof. In some further embodiments, L¹ comprises

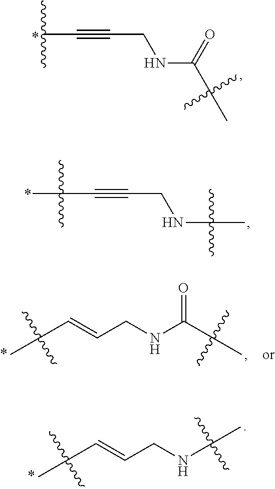

In some further embodiments, the asterisk * indicates the point of attachment of L¹ to the nucleobase (e.g., C₅ position of a pyrimidine base or the C7 position of a 7-deaza purine base).

Some further embodiments of the nucleoside or nucleotide described herein include those with Formula (Ia), (Ia′), (Ib), (Ic), (Ic′) or (Id):

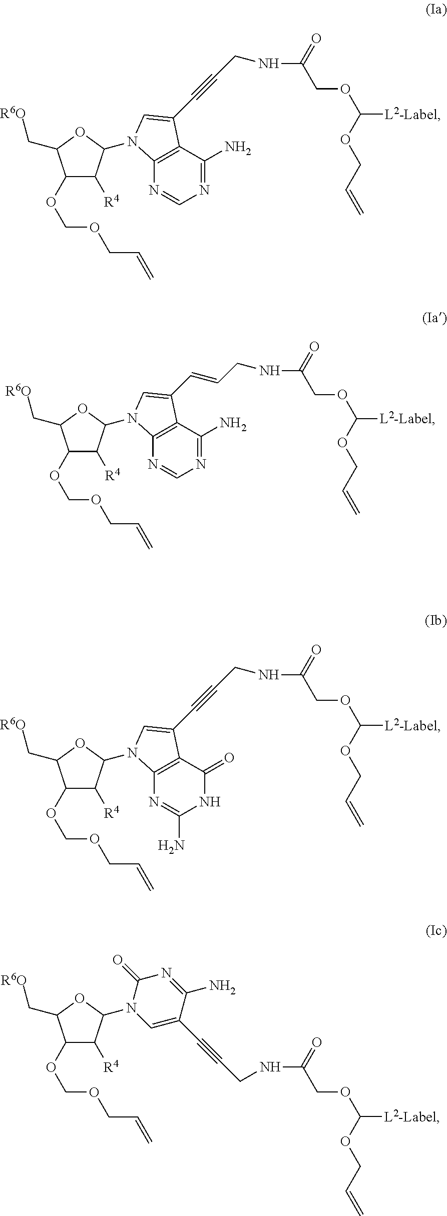

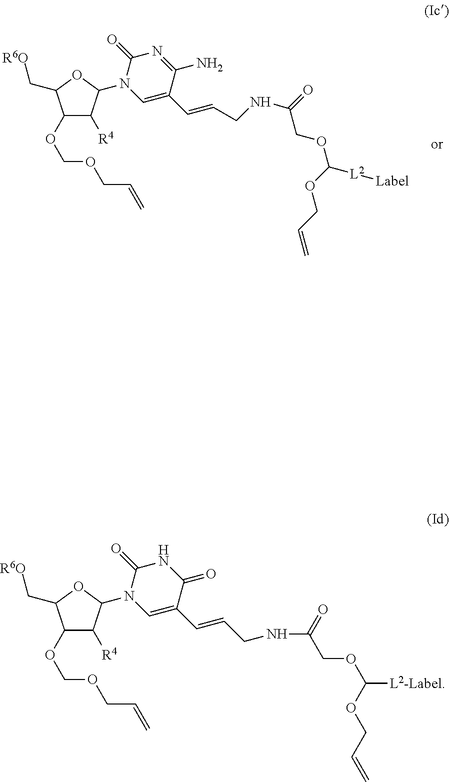

In some further embodiments of the nucleotide described herein, L² is present and L² comprises

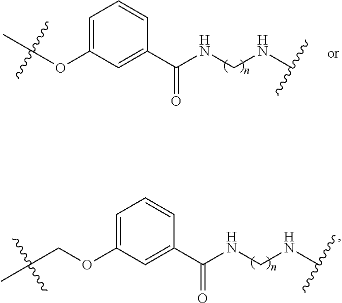

wherein n is an integer of 1, 2, 3, 4, 5, 6, 7, 8, 9 or 10 and the phenyl moiety is optionally substituted. In some such embodiments, n is 5 and the phenyl moiety of L² is unsubstituted.

In any embodiments of the nucleotide described herein, the cleavable linker or L¹/L² may further comprise a disulfide moiety or azido moiety (such as

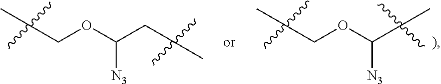 [SMILES: *COC(C*)N=[N+]=[N-] ; *COC(*)N=[N+]=[N-]]

or a combination thereof. Additional non-limiting examples of a linker moiety may be incorporated into L¹ or L² include:

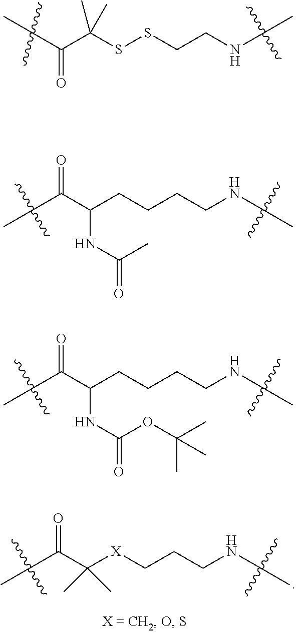

Additional linker moieties are disclosed in WO 2004/018493 and U.S. Publication Nos. 2016/0040225 and 2021/0403500, which are herein incorporated by references.

Non-limiting exemplary labeled nucleotides as described herein include:

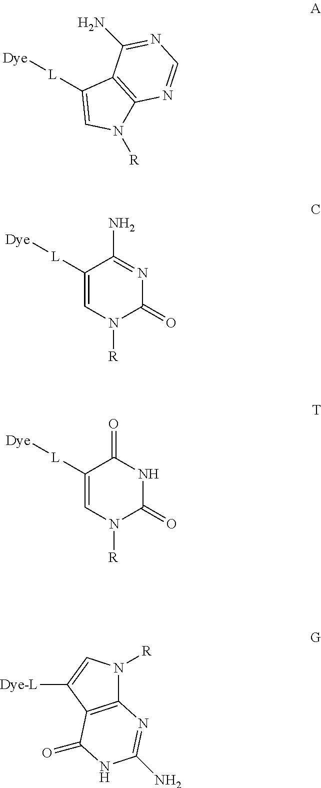

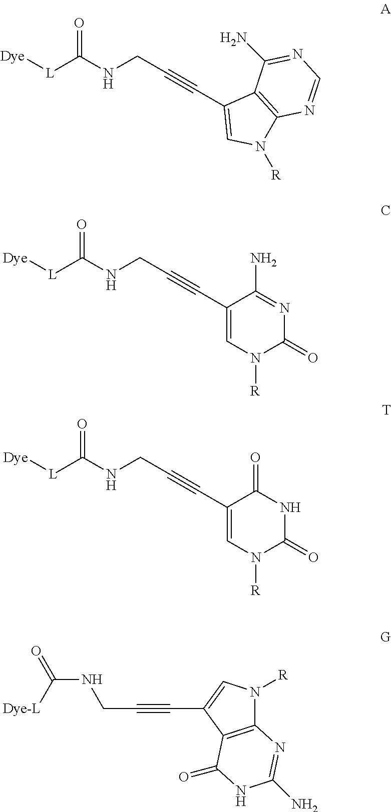

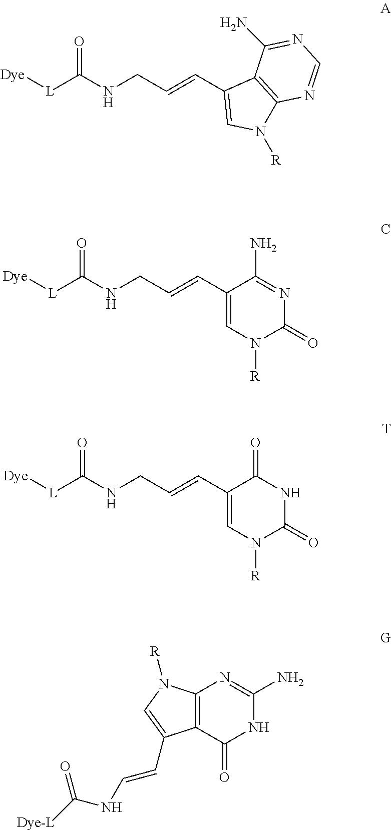

wherein L represents a cleavable linker (optionally include L² described herein) and R represents a ribose or deoxyribose moiety as described above, or a ribose or deoxyribose moiety with the 5′ position substituted with one, two or three phosphates.

In some embodiments, non-limiting exemplary fluorescent dye conjugates are shown below:

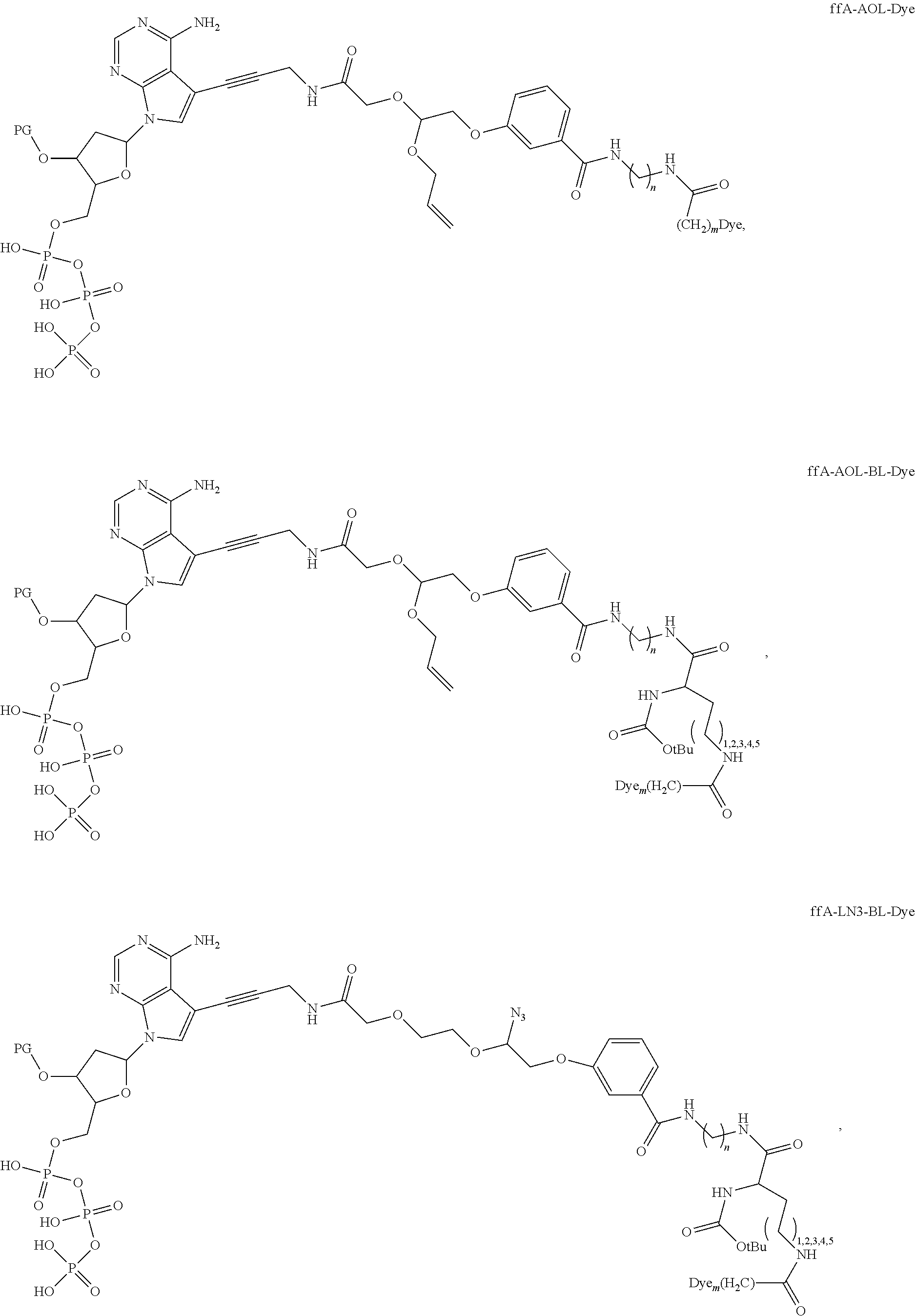

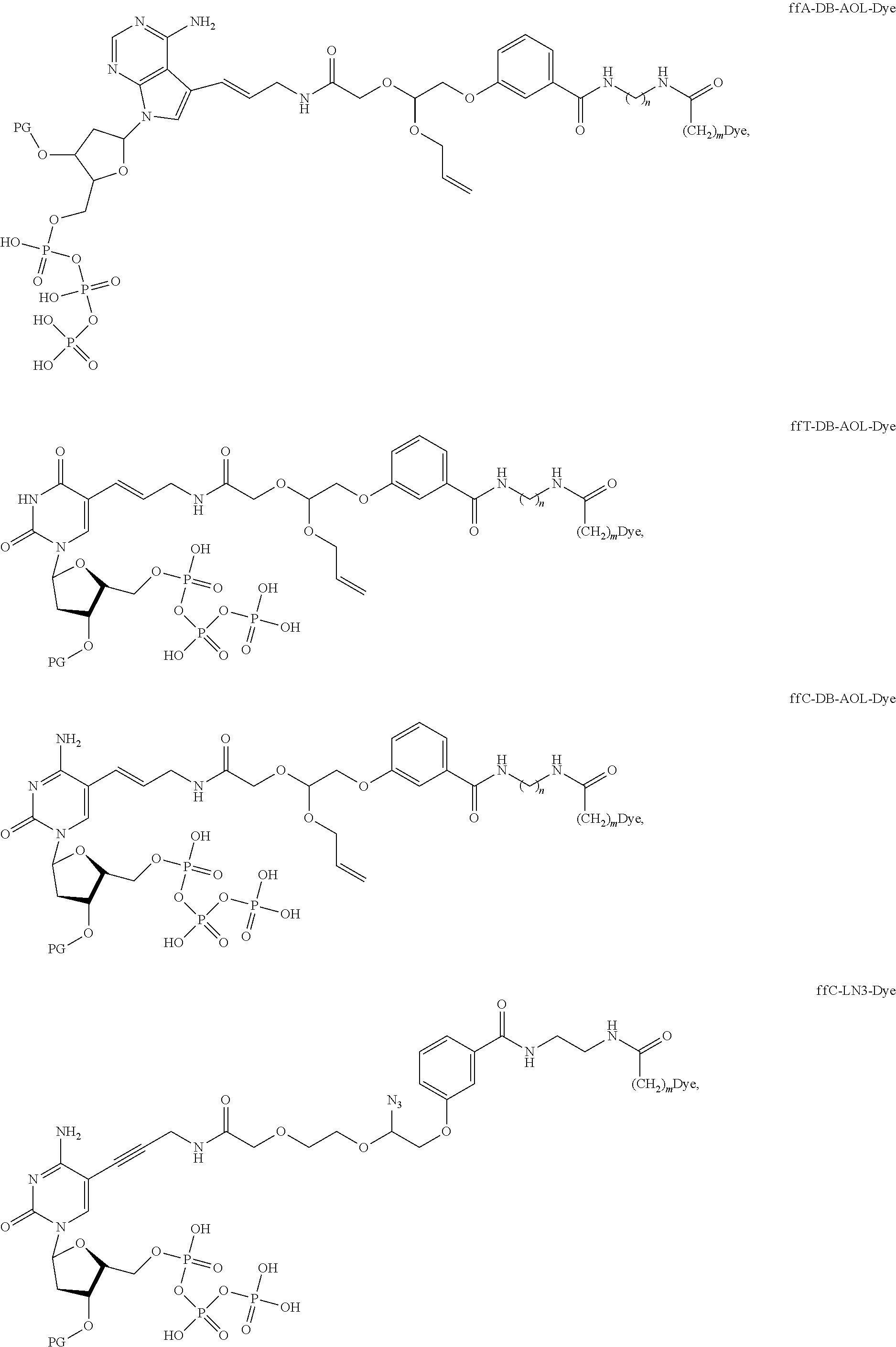

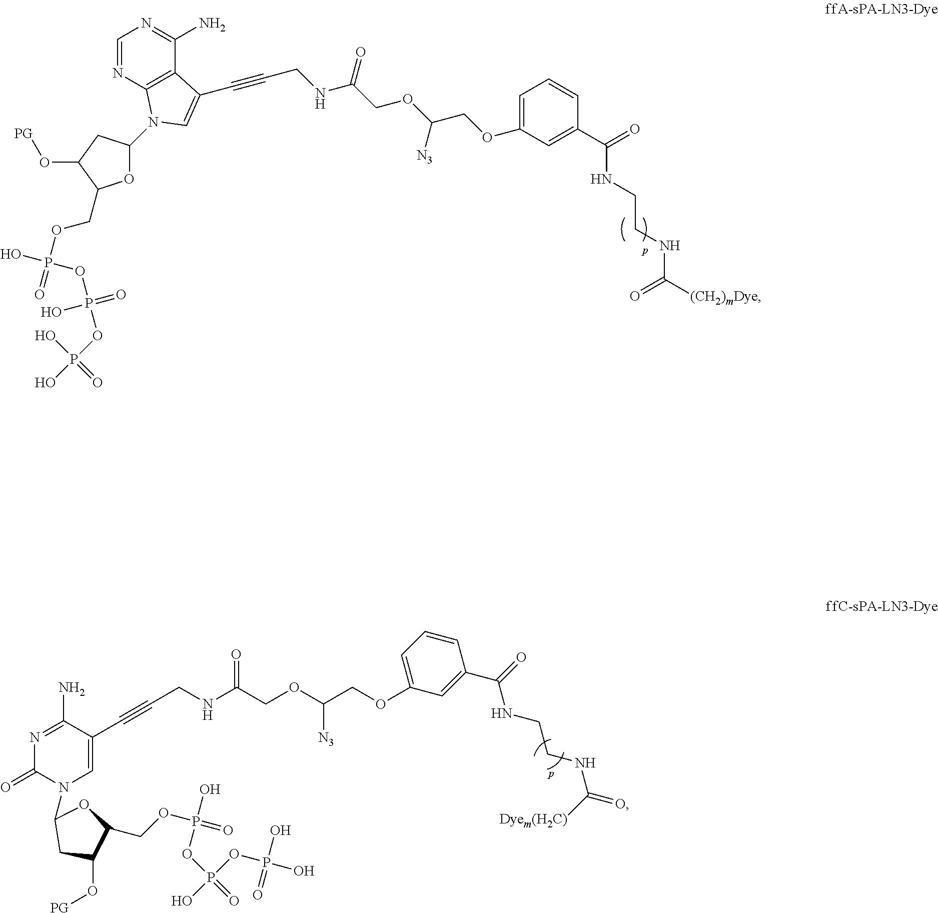

wherein PG stands for the 3′ blocking groups described herein; each of n and p is an integer of 1, 2, 3, 4, 5, 6, 7, 8, 9, or 10; and m is 0, 1, 2, 3, 4, or 5. In one embodiment, —O-PG is AOM. In another embodiment, —O-PG is —O-azidomethyl (AZM). In one embodiment n is 2. In one embodiment, p is 1. In one embodiment, m is 5.

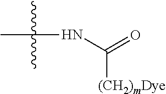

refers to the connection point of the Dye with the cleavable linker as a result of a reaction between an amino group of the linker moiety and the carboxyl group of the Dye. In some embodiments, the short pendant arm (sPA) linker is also referred to as sPA-LN3 linker.

Various fluorescent dyes may be used in the present disclosure as detectable labels, in particularly those dyes that may be excited by a blue light (e.g., about 450 nm to about 460 nm) or a green light (e.g., about 520 nm to about 540 nm). These dyes may also be referred to as “blue dyes” and “green dyes” respectively. Examples of various type of blue dyes, including but not limited to coumarin dyes, chromenoquinoline dyes, bisboron containing heterocycles, and naphthalimide dyes that are disclosed in U.S. Publication Nos. 2018/0094140, 2018/0201981, 2020/0277529, 2020/0277670, 2021/0188832, 2022/0033900, 2022/0195196, 2022/0195517, 2022/0380389, 2023/0313292, and 2023/0416279, and U.S. Ser. Nos. 63/492,896 and 63/593,489, each of which is incorporated by reference in its entirety. Examples of green dyes including cyanine or polymethine dyes disclosed in International Publication Nos. WO2013/041117, WO2014/135221, WO 2016/189287, WO2017/051201 and WO2018/060482A1, as well as U.S. Ser. No. 63/616,289, each of which is incorporated by reference in its entirety.

In any embodiments of nucleotide described herein, the nucleotide comprises a 2′ deoxyribose moiety (i.e., R⁴ is Formula (I) and (Ia)-(Id)) is H). In some further respect, the 2′ deoxyribose contains one, two or three phosphate groups at the 5′ position of the sugar ring. In some further aspect, the nucleotides described herein are nucleotide triphosphate (i.e., —OR⁶ in Formula (I) and (Ia)-(Id)) is triphosphate).

Additional embodiments of the present disclosure relate to an oligonucleotide or a polynucleotide comprising a nucleoside or nucleotide described herein. In some such embodiments, the oligonucleotide or polynucleotide is hybridized to a template or target polynucleotide. In some such embodiments, the template polynucleotide is immobilized on a solid support.

Additional embodiments of the present disclosure relate to a solid support comprises an array of a plurality of immobilized template or target polynucleotides and at least a portion of such immobilized template or target polynucleotides is hybridized to an oligonucleotide or a polynucleotide comprising a nucleoside or nucleotide described herein.

The present application will also be further described with reference to DNA, although the description will also be applicable to RNA, PNA, and other nucleic acids, unless otherwise indicated.

### Embodiments and Alternatives of Sequencing-By-Synthesis

Alternatively, the sequencing methods described herein may also be carried out using unlabeled nucleotides and affinity reagents containing a fluorescent dye described herein. For example, one, two, three or each of the four different types of nucleotides (e.g., dATP, dCTP, dGTP and dTTP or dUTP) in the incorporation mixture of step (a) may be unlabeled. Each of the four types of nucleotides (e.g., dNTPs) has a 3′ blocking group to ensure that only a single base can be added by a polymerase to the 3′ end of the primer polynucleotide. After incorporation of an unlabeled nucleotide in step (b), the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:
(a′) contacting a solid support with an incorporation mixture comprising DNA polymerase and one or more of four different types of unlabeled nucleotides (e.g., dATP, dCTP, dGTP, and dTTP or dUTP), wherein the solid support comprises a plurality of different target polynucleotides immobilized thereon, and sequencing primers that are complementary and hybridized to at least a portion of the target polynucleotides;
(b′) incorporating one type of nucleotides into the sequencing primers to produce extended copy polynucleotides, wherein each of the four types of nucleotides comprises a 3′ blocking group;
(c′) contacting the extended copy polynucleotides with a set of affinity reagents under conditions wherein one affinity reagent binds specifically to the incorporated unlabeled nucleotides to provide labeled extended copy polynucleotides;
(d′) imaging the solid support and performing one or more fluorescent measurements of the extended copy polynucleotides; and
(e′) removing the 3′ blocking group of the incorporated nucleotides;
wherein the aqueous cleavage solution comprises one or more additives for improving thermal or oxidative stability of the active palladium catalyst, and wherein the one or more additives comprise one or more water soluble macrocycles as described herein. In some embodiments, the additives in aqueous cleavage solution further comprise one or more oxygen scavengers and/or phosphine reducing agents as described herein.

In some embodiments of the modified sequencing method described herein, the method further comprises removing the affinity reagents from the incorporated nucleotides. In still further embodiments, the 3′ blocking group and the affinity reagent are removed in the same reaction. In some embodiments, the method further comprises a step (f′) washing the solid support with an aqueous wash solution. In further embodiments, steps (a′) through (f′) are repeated at least 50, 100, 150, 200, 250, 300, 350, 400, 450 or 500 cycles to determine the target polynucleotide sequences. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable labeled that is spectrally distinguishable. In some embodiments, the affinity reagents may include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, at least one affinity reagent is an antibody or a protein tag. In another embodiment, at least one of the first type, the second type, and the third type of affinity reagents is an antibody or a protein tag comprising one or more detectable labels (e.g., multiple copies of the same detectable label), wherein the detectable label is or comprises a bis-boron dye moiety described herein.

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina, Inc.), and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

Some embodiments can utilize detection of four different nucleotides using fewer than four different labels. For example, SBS can be performed utilizing methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis,” Acc. Chem. Res. 35:817-825 (2002); Li, J., M. Gershow, D. Stein, E. Brandin, and J. A. Golovchenko, “DNA molecules and configurations in a solid-state nanopore microscope,” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores,” Clin. Chem. 53, 1996-2001 (2007); Healy, K. “Nanopore-based single-molecule DNA analysis,” Nanomed. 2, 459-481 (2007); Cockroft, S. L., Chu, J., Amorin, M. & Ghadiri, M. R. “A single-molecule nanopore device detects DNA polymerase activity with single-nucleotide resolution,” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some other embodiments of sequencing method involve the use the 3′ blocked nucleotide described herein in nanoball sequencing technique, such as those described in U.S. Pat. No. 9,222,132, the disclosure of which is incorporated by reference. Through the process of rolling circle amplification (RCA), a large number of discrete DNA nanoballs may be generated. The nanoball mixture is then distributed onto a patterned slide surface containing features that allow a single nanoball to associate with each location. In DNA nanoball generation, DNA is fragmented and ligated to the first of four adapter sequences. The template is amplified, circularized and cleaved with a type II endonuclease. A second set of adapters is added, followed by amplification, circularization and cleavage. This process is repeated for the remaining two adapters. The final product is a circular template with four adapters, each separated by a template sequence. Library molecules undergo a rolling circle amplification step, generating a large mass of concatemers called DNA nanoballs, which are then deposited on a flow cell. Goodwin et al., “Coming of age: ten years of next-generation sequencing technologies,” Nat Rev Genet. 2016; 17 (6): 333-51.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations,” Science 299, 682-686 (2003); Lundquist, P. M. et al. “Parallel confocal detection of single molecules in real time,” Opt. Lett. 33, 1026-1028 (2008); Korlach, J. et al. “Selective aluminum passivation for targeted immobilization of single DNA polymerase molecules in zero-mode waveguide nano structures,” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617, all of which are incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in U.S. Pub. No. 2010/0111768 and U.S. patent application Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. patent application Ser. No. 13/273,666, which is incorporated herein by reference.

Arrays in which polynucleotides have been directly attached to silica-based supports are those for example disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported template polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178 and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly (N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Templates that are to be sequenced may form part of an “array” on a solid support, in which case the array may take any convenient form. Thus, the method of the disclosure is applicable to all types of high-density arrays, including single-molecule arrays, clustered arrays, and bead arrays. Labeled nucleotides of the present disclosure may be used for sequencing templates on essentially any type of array, including but not limited to those formed by immobilization of nucleic acid molecules on a solid support.

However, labeled nucleotides of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using the nucleotides labeled with dye compounds of the disclosure.

The labeled nucleotides of the present disclosure are also useful in sequencing of templates on single molecule arrays. The term “single molecule array” (“SMA”) as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photo-bleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the nucleotides of the disclosure is in sequencing-by-synthesis reactions, the utility of the nucleotides is not limited to such methods. In fact, the nucleotides may be used advantageously in any sequencing methodology which requires detection of fluorescent labels attached to nucleotides incorporated into a polynucleotide.

In particular, the labeled nucleotides of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses labeled nucleotides which are dideoxynucleotides lacking hydroxy groups at both of the 3′ and 2′ positions, such dideoxynucleotides being suitable for use in Sanger type sequencing methods and the like.

Labeled nucleotides of the present disclosure incorporating 3′ blocking groups, it will be recognized, may also be of utility in Sanger methods and related protocols since the same effect achieved by using dideoxy nucleotides may be achieved by using nucleotides having 3′-OH blocking groups: both prevent incorporation of subsequent nucleotides. Where nucleotides according to the present disclosure, and having a 3′ blocking group are to be used in Sanger-type sequencing methods it will be appreciated that the dye compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

### Methods for Enhancing Intensity of a Dye

Additional aspects of the present disclosure relates to a method for enhancing fluorescent signal intensity of a fluorescent dye during an imaging event, comprising:

contacting the fluorescent dye with an aqueous scan mixture composition comprising one or more water-soluble macrocycles, wherein the water-soluble macrocycle encapsulates the fluorescent dye to form a host-guest complex with the fluorescent dye. In some embodiments, the method also reduces fluorescent signal decay of a fluorescent dye during an imaging event.

In some embodiments of the method described herein, the water-soluble macrocycle comprises water-soluble cyclodextrins, water-soluble calixarenes, water-soluble cucurbiturils, or optionally substituted analogs, salts, or hydrates thereof, or combination thereof. In further embodiments, the water-soluble cyclodextrins or the optionally substituted analogs, salts, or hydrates thereof comprise α-cyclodextrin, β-cyclodextrin, γ-cyclodextrin, or substituted analogs or salts thereof, or combination thereof. In further embodiments, the water-soluble cyclodextrin or the substituted analogs, salts, or hydrates thereof comprise or are selected from the group consisting of α-cyclodextrin, β-cyclodextrin, (2-hydroxypropl)-β-cyclodextrin, acetyl-β-cyclodextrin, (2-hydroxyethyl)-β-cyclodextrin, heptakis (2,3,6-tri-O-methyl)-β-cyclodextrin, succinyl-β-cyclodextrin, methyl-β-cyclodextrin, carboxymethyl-β-cyclodextrin, heptakis (6-deoxy-6-amino)-β-cyclodextrin heptahydrochloride, γ-cyclodextrin hydrate, (2-hydroxypropyl)-γ-cyclodextrin, and salts and combinations thereof. In yet further embodiments, the water-soluble cyclodextrin comprises or is (2-hydroxypropyl)-β-cyclodextrin or methyl-β-cyclodextrin. In some embodiments, the binding affinity constant of the water-soluble cyclodextrin or optionally substituted analog, salt, or hydrate thereof to the dye is at least about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,500, 3,000, 3,500, 4,000, 4,500, or 5,000M⁻¹ or in a range defined by any two of the preceding values. In certain embodiments, the affinity constant (Kb) of the water-soluble cyclodextrin, or optionally substituted analog, salt, or hydrate thereof to the detectable label may be at least about 100, 200, 300, 400, or 500M⁻¹. In some embodiments, the concentration of water soluble cyclodextrin or the optionally substituted analog, salt, or hydrate thereof in the scan mixture is about 0.1, 0.5, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750 775, 800, 825, 850, 875, 900, 925, 950, 975, 1,000, 2,000, 3,000, 4,000, 5,000 mM or in a range defined by any two of the preceding values. In some further embodiments, the concentration of water-soluble cyclodextrin or the optionally substituted analog, salt, or hydrate thereof in the scan mixture is about 0.1 mM to about 500 mM, about 0.2 mM to about 400 mM, about 0.4 mM to about 200 mM, about 0.6 mM to about 100 mM, about 0.8 mM to about 50 mM, about 1 mM to about 10 mM, about 2 mM to about 8 mM, about 4 mM to about 6 mM, or about 5 mM. In some further embodiments, the concentration of the water-soluble cyclodextrin or the optionally substituted analog, salt, or hydrate thereof in the aqueous scan mixture is from about 1 mM to about 500 mM, about 5 mM to about 400 mM, about 10 mM to about 300 mM, or about 20 mM to about 150 mM.

In some embodiments of the method described herein, the water-soluble cucurbituril or the optionally substituted analogs, salts, or hydrates thereof comprise cucurbituril hydrates or substituted analogs or salts thereof, or combination thereof. In some embodiments, the cucurbituril hydrates comprise or are selected from the group consisting of cucurbit [5]uril hydrate, cucurbit [6]uril hydrate, cucurbit [7]uril hydrate, cucurbit [8]uril hydrate, or optionally substituted analogs or salts thereof. In some embodiments, the binding affinity constant of the water-soluble cucurbituril to the dye is at least about 1,000, 10,000, 25,000, 50,000, 75,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, 1,000,000, 2,000,000, 3,000,000, 4,000,000, or 5,000,000M⁻¹ or in a range defined by any two of the preceding values. In certain embodiments, the binding affinity constant of the water-soluble cucurbituril to the dye is at least about 1×10⁴M⁻¹, 2×10⁴M⁻¹, 3×10⁴M⁻¹, 4×10⁴M⁻¹, 5×10⁴M⁻¹, 6×10⁴M⁻¹, 7×10⁴M⁻¹, 8×10⁴M⁻¹, 9×10⁴M⁻¹, 1×10⁵M⁻¹, 2×10⁵M⁻¹, or 3×10⁵M⁻¹. In some further embodiments, the binding affinity constant of the water-soluble cucurbituril to the detectable label is at least about 1×10⁵M⁻¹. In some embodiments, the concentration of water-soluble cucurbituril or the optionally substituted analog, salt, or hydrate thereof is about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, or 50 mM, or in a range defined by any two of the preceding values. In some further embodiments, the concentration of water soluble cucurbituril or the optionally substituted analog, salt, or hydrate thereof in the scan mixture is about 0.001 mM to about 1 mM, about 0.005 mM to about 0.5 mM, about 0.01 mM to about 0.1 mM, about 0.02 mM to about 0.08 mM, about 0.04 mM to about 0.06 mM, or about 0.05 mM.

In some embodiments of the method described herein, the water-soluble calixarenes or the optionally substituted analogs, salts, or hydrates thereof comprise sulfocalixarenes or substituted analogs or salts thereof, or combination thereof. In some embodiments, the water-soluble calixarenes or the substituted analogs, salts, or hydrates thereof comprise or are selected from the group consisting of 4-sulfocalix [4] arene, 4-sulfocalix [4] arene hydrate, 4-sulfothiacalix [4] arene, and salts and combinations thereof. In some embodiments, the binding affinity constant of the water-soluble calixarene to the detectable label is at least about 500, 1000, 1500, 2000, 2500, 3000, 3500, 4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19000, 20,000 or 25,000, or in a range defined by any two of the preceding values. In some embodiments, the concentration of water-soluble calixarene or the optionally substituted analog, salt, or hydrate thereof in the scan mixture is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, or 500 mM or in a range defined by any two of the preceding values. In some further embodiments, the concentration of water soluble calixarene or the optionally substituted analog, salt, or hydrate thereof in the scan mixture is about 0.01 mM to about 10 mM, about 0.05 mM to about 5 mM, about 0.1 mM to about 1 mM, about 0.2 mM to about 0.8 mM, about 0.4 mM to about 0.6 mM, or about 0.5 mM.

In some embodiments of the method described herein, the one or more water-soluble macrocycles described herein increases or enhances the fluorescence of the dye by at least 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 225%, 250%, 275%, or 300% compared to the fluorescence of the dye in the same scan mixture without the one or more water-soluble macrocycles. In some embodiments, the imaging event uses a light source having a wavelength from about 400 to about 550 nm, from about 450 nm to about 460 nm, from about 520 nm to about 540 nm, or from about 490 nm to 520 nm. In some further embodiments, the imaging event uses two different light sources having two different wavelengths, for example, from about 450 nm to about 460 nm, and from about 520 nm to about 540 nm. In some embodiments, the dye is covalently bound to a nucleotide, or a polynucleotide. In some embodiments, the dye is bound to a polynucleotide immobilized onto a surface of a solid support.

### Kits

A kit for use with a sequencing apparatus, comprising:
an incorporation mixture comprising one or more different types of nucleotides, wherein at least one nucleotide is labeled with a detectable label;
an aqueous scan mixture comprising one or more water-soluble macrocycles. In some embodiments, the water-soluble macrocycle is capable of encapsulating the detectable label (i.e., to form a host-guest complex with the detectable label).

In some embodiments of the method described herein, the water-soluble macrocycle comprises water-soluble cyclodextrins, water-soluble calixarenes, water-soluble cucurbiturils, or optionally substituted analogs, salts (e.g., an acid addition salt such as HCl salt, or an alkali metal salt such as KCl or NaCl), or hydrates thereof, or combination thereof. In further embodiments, the water-soluble cyclodextrins or the optionally substituted analogs, salts, or hydrates thereof comprise α-cyclodextrin, β-cyclodextrin, γ-cyclodextrin, or substituted analogs or salts thereof, or combination thereof. In further embodiments, the water-soluble cyclodextrin or the substituted analogs, salts, or hydrates thereof comprise or are selected from the group consisting of α-cyclodextrin, β-cyclodextrin, (2-hydroxypropl)-β-cyclodextrin, acetyl-β-cyclodextrin, (2-hydroxyethyl)-β-cyclodextrin, heptakis (2,3,6-tri-O-methyl)-β-cyclodextrin, succinyl-β-cyclodextrin, methyl-β-cyclodextrin, carboxymethyl-β-cyclodextrin, heptakis (6-deoxy-6-amino)-β-cyclodextrin heptahydrochloride, γ-cyclodextrin hydrate, (2-hydroxypropyl)-γ-cyclodextrin, and salts and combinations thereof. In yet further embodiments, the water-soluble cyclodextrin comprises or is (2-hydroxypropyl)-β-cyclodextrin or methyl-β-cyclodextrin. In some embodiments, the binding affinity constant of the water-soluble cyclodextrin or optionally substituted analogs, salts, or hydrates thereof to the dye is at least about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, 1,800, 1,825, 1,850, 1,875, 1,900, 1,925, 1,950, 1,975, 2,000, 2,500, 3,000, 3,500, 4,000, 4,500, or 5,000M⁻¹ or in a range defined by any two of the preceding values. In certain embodiments, the affinity constant (Kb) of the water-soluble cyclodextrin, or optionally substituted analogs, salts, or hydrates thereof to the detectable label may be at least about 100, 200, 300, 400, or 500M⁻¹. In some embodiments, the concentration of water soluble cyclodextrin or the optionally substituted analogs, salts, or hydrates thereof in the scan mixture is about 0.1, 0.5, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750 775, 800, 825, 850, 875, 900, 925, 950, 975, 1,000, 2,000, 3,000, 4,000, 5,000 mM or in a range defined by any two of the preceding values. In some further embodiments, the concentration of water-soluble cyclodextrin or the optionally substituted analogs, salts, or hydrates thereof in the scan mixture is about 0.1 mM to about 500 mM, about 0.2 mM to about 400 mM, about 0.4 mM to about 200 mM, about 0.6 mM to about 100 mM, about 0.8 mM to about 50 mM, about 1 mM to about 10 mM, about 2 mM to about 8 mM, about 4 mM to about 6 mM, or about 5 mM. In some further embodiments, the concentration of the water-soluble cyclodextrin or the optionally substituted analog, salt, or hydrate thereof in the aqueous scan mixture is from about 1 mM to about 500 mM, about 5 mM to about 400 mM, about 10 mM to about 300 mM, or about 20 mM to about 150 mM.

In some embodiments of the method described herein, the water-soluble cucurbituril or the optionally substituted analogs, salts, or hydrates thereof comprise cucurbituril hydrates or substituted analogs or salts thereof, or combination thereof. In some embodiments, the cucurbituril hydrates comprise or are selected from the group consisting of cucurbit [5]uril hydrate, cucurbit [6]uril hydrate, cucurbit [7]uril hydrate, cucurbit [8]uril hydrate, or optionally substituted analogs or salts thereof. In some embodiments, the binding affinity constant of the water-soluble cucurbituril to the dye is at least about 1,000, 10,000, 25,000, 50,000, 75,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, 1,000,000, 2,000,000, 3,000,000, 4,000,000, or 5,000,000M⁻¹ or in a range defined by any two of the preceding values. In certain embodiments, the binding affinity constant of the water-soluble cucurbituril to the dye is at least about 1×10⁴M⁻¹, 2×10⁴M⁻¹, 3×10⁴M⁻¹, 4×10⁴M⁻¹, 5×10⁴M⁻¹, 6×10⁴M⁻¹, 7×10⁴M⁻¹, 8×10⁴M⁻¹, 9×10⁴M⁻¹, 1×10⁵M⁻¹, 2×10⁵M⁻¹, or 3×10⁵M⁻¹. In some further embodiments, the binding affinity constant of the water-soluble cucurbituril to the detectable label is at least about 1×10⁵M⁻¹. In some embodiments, the concentration of water-soluble cucurbituril or the optionally substituted analog, salt, or hydrate thereof is about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, or 50 mM, or in a range defined by any two of the preceding values. In some further embodiments, the concentration of water soluble cucurbituril or the optionally substituted analog, salt, or hydrate thereof in the scan mixture is about 0.001 mM to about 1 mM, about 0.005 mM to about 0.5 mM, about 0.01 mM to about 0.1 mM, about 0.02 mM to about 0.08 mM, about 0.04 mM to about 0.06 mM, or about 0.05 mM.

In some embodiments of the method described herein, the water-soluble calixarenes or the optionally substituted analogs, salts, or hydrates thereof comprise sulfocalixarenes or substituted analogs or salts thereof, or combination thereof. In some embodiments, the water-soluble calixarenes or the substituted analogs, salts, or hydrates thereof comprise or are selected from the group consisting of 4-sulfocalix [4] arene, 4-sulfocalix [4] arene hydrate, 4-sulfothiacalix [4] arene, and optionally substituted analogs and salts and combinations thereof. In some embodiments, the binding affinity constant of the water-soluble calixarene to the detectable label is at least about 500, 1000, 1500, 2000, 2500, 3000, 3500, 4000, 4500, 5000, 5500, 6000, 6500, 7000, 7500, 8000, 8500, 9000, 9500, 10,000, 11,000, 12,000, 13,000, 14,000, 15,000, 16,000, 17,000, 18,000, 19000, 20,000 or 25,000, or in a range defined by any two of the preceding values. In some embodiments, the concentration of water-soluble calixarene or the optionally substituted analog, salt, or hydrate thereof in the scan mixture is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, or 500 mM or in a range defined by any two of the preceding values. In some further embodiments, the concentration of water soluble calixarene or the optionally substituted analog, salt, or hydrate thereof in the scan mixture is about 0.01 mM to about 10 mM, about 0.05 mM to about 5 mM, about 0.1 mM to about 1 mM, about 0.2 mM to about 0.8 mM, about 0.4 mM to about 0.6 mM, or about 0.5 mM.

In some embodiments, the incorporation mixture comprises at least some fully functional nucleotides (ffNs). In some embodiments, the one or more of four different types of nucleotides comprises A, C, G and T or U, and at least one type of nucleotides also carries a detectable label. In some embodiments, the one or more of four different types of nucleotides comprises dA, dC, dG and dT or dU, and at least one type of nucleotides also carries a detectable label.

The compounds, nucleotides, or kits that are set forth herein may be used to detect, measure, or identify a biological system (including, for example, processes or components thereof). Exemplary techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two light sources operating at different wavelengths.

In a particular embodiment, the labeled nucleotide(s) described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, nucleotides, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample. When two nucleotides labeled with fluorescent dye compounds are supplied in kit form, it is a feature of some embodiments that the spectrally distinguishable fluorescent dyes can be excited at the same wavelength, such as, for example by the same light source. When four nucleotides labeled with fluorescent dye compounds are supplied in kit form, it is a feature of some embodiments that two of the spectrally distinguishable fluorescent dyes can both be excited at one wavelength and the other two spectrally distinguishable dyes can both be excited at another wavelength. Particular excitation wavelengths for the dyes are between 450-460 nm, 490-520 nm, or 520 nm or above (e.g., 532 nm).

Although kits are exemplified herein in regard to configurations having different nucleotides that are labeled with different dye compounds, it will be understood that kits can include 2, 3, 4 or more different nucleotides that have the same dye compound.

In addition to the labeled nucleotides, the kit may comprise together at least one additional component. The further component(s) may be one or more of the components identified in a method set forth herein or in the Examples section below. Some non-limiting examples of components that can be combined into a kit of the present disclosure are set forth below. In some embodiments, the kit further comprises a DNA polymerase (such as a mutant of 9°N polymerase, such as those disclosed in WO 2005/024010, U.S. Publication Nos. 2020/0131484 A1, 2020/0181587 A1, and U.S. Ser. Nos. 63/412,241 and 63/433,971, each of which is incorporated by reference herein in its entirety) and one or more buffer compositions. One buffer composition may comprise antioxidants such as ascorbic acid or sodium ascorbate, which can be used to protect the dye compounds from photo damage during detection. Additional buffer composition may comprise a reagent can may be used to cleave the 3′ blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure. In any embodiments of the nucleotide or labeled nucleotide described herein, the nucleotide contains a 3′ blocking group.

Alternatively, the kit may comprise one or more different types of unlabeled 3′ blocked nucleotide and one or more affinity reagents (e.g., protein tags and antibodies).

### EXAMPLES

Additional embodiments are disclosed in further detail in the following examples, which are not in any way intended to limit the scope of the claims.

### Example 1. Effect of Macrocycles on Fluorescence and UV Signal

In a first experiment, 21 commercially available water-soluble macrocycles were screened for their effects on dye fluorescence. In an aqueous solution, each macrocycle was added to a solution of ffC-LN3-coumarin dye A in 10 mM Tris (pH=8.0). Coumarin dye A has the structure:

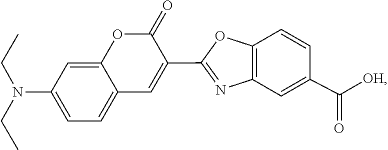 [SMILES: CCN(CC)c1ccc2cc(-c3nc4cc(C(=O)O)ccc4o3)c(=O)oc2c1]

ffC-LN3-coumarin dye A is disclosed in U.S. Publication No. 2020/0277670 A1, which is incorporated by reference in its entirety. Coumarin dye A is a blue dye that is excitable by a light source having a wavelength from about 450 nm to about 460 nm. The concentration of the ffC-LN3-coumarin dye A in the solution is about 5 μM. The concentration of macrocycle added to obtain this signal variation were 5.0 mM for cyclodextrins (i.e., M1-M14), 0.5 mM for calixarenes (i.e., M16-M18), and 0.05 mM for cucurbiturils (i.e., M19-M22). The UV-Vis and fluorescence intensity values were monitored after the macrocycle addition. The data were analyzed to obtain the % signal variation for florescence and UV-Vis intensity and the affinity constant were interpolated using BindSim™. The percentage variation of UV-Vis and fluorescent signal intensity were calculated, and the results are summarized in FIG. 2A. FIG. 2A shows normalized change in signal (% ASignal/Signalo) for fluorescence intensity (fluorescence variation) and UV-Vis absorption (UV variation) at the maximum dye wavelength. It can be observed that there was a variability in quenching or enhancement of the fluorescence signal depending on the macrocycle used and the functional group present in the molecule. For example, the cyclodextrins showed both properties of quenching and signal enhancement in solution, and the cucurbiturils showed minor fluorescence variation except for M21 (CB7), which provided a strong fluorescence enhancement.

In a second experiment, a set of the macrocycles (i.e., M3, M4, M5, M8, M9, M10, M14, M17, M21, M22) were chosen for testing in both a Tris buffer containing ffC-LN3-coumarin dye A and a scan mixture containing ffC-LN3-coumarin dye A. The scan mixture included Tris pH 7.4 (1M), 0.05% Tween 20, sodium ascorbate (20 mM), and hydroxy ethyl gallate (10 mM). The concentration of macrocycle added to obtain this signal variation were 5.0 mM for cyclodextrins (i.e., M3, M4, M5, M8, M9, M10, M14), 0.5 mM for calixarenes (i.e., M17), and 0.05 mM for cucurbiturils (i.e., M21 and M22). The results of this test are plotted in FIG. 2B, which shows normalized change in signal (% ASignal/Signalo) for fluorescence intensity in tris and in scan mixture at the maximum dye wavelength. The results show that the macrocycles exhibit similar properties in terms of fluorescence enhancement of the ffC when added to a scan mixture.

### Example 2. Binding Affinity Constant of Macrocycles to Dye

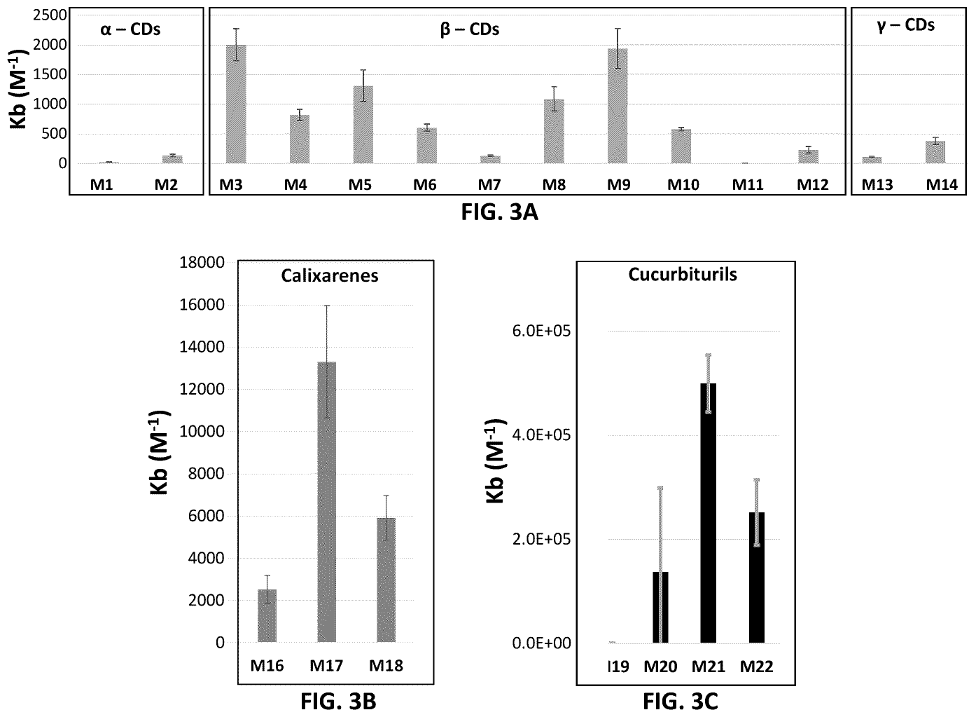

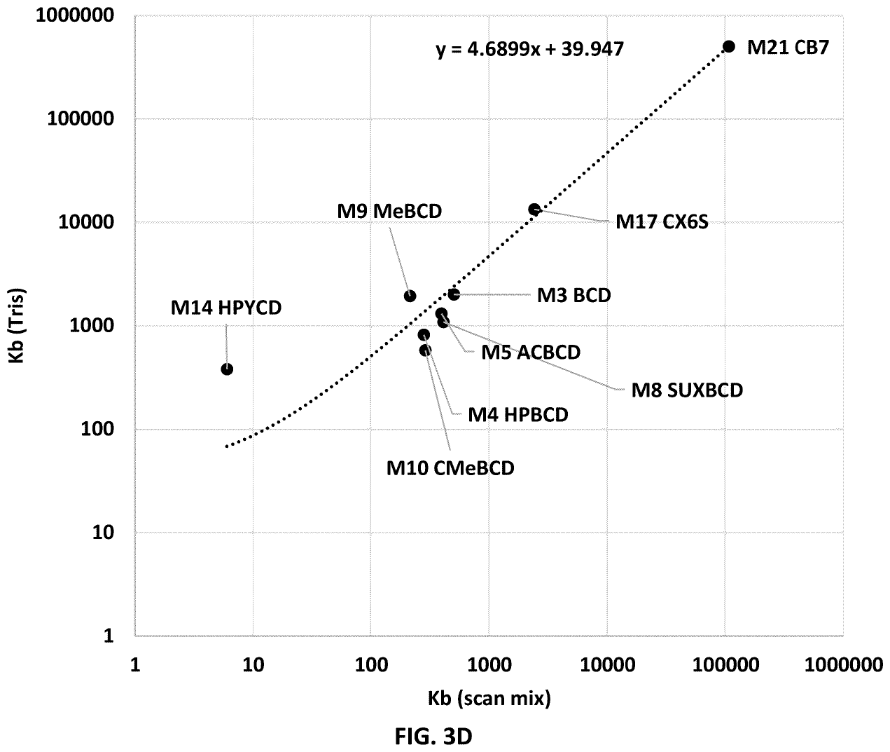

To evaluate affinity of each macrocycle to ffC-LN3-coumarin dye A, ffC-LN3-coumarin dye A fluorescence and UV-Vis maximum intensity values were measured and plotted against the concentration of each macrocycle added. These data were fitted to a 1:1 binding isotherm to calculate the binding affinity constant (Kb) for each macrocycle to coumarin dye A in the Tris buffer. FIGS. 3A-3C illustrate these calculated affinity constants for the macrocycles to coumarin dye A. The binding process was also combined with blue shift of the maximum dye absorption/emission which agrees with a change of the local environment surrounding the dye, due to the encapsulation process. It was observed that the binding affinity constant (Kb) range for cyclodextrins, as shown in FIG. 3A, can vary from 10 to 2000M⁻¹. Fluorescence intensity variation do not correlate with higher affinity.

In another experiments, the binding affinity of a set of macrocycles (i.e., M3, M4, M5, M8, M9, M10, M14, M17, M21, M22) to ffC-LN3-coumarin dye A in scan mixture was also calculated using similar method as described above and the comparative results are shown in FIG. 3D. The results show that the macrocycles exhibit similar properties in terms of affinity values to the ffC when added to a scan mixture.

### Example 3. Experiments Measuring Photobleaching and Singlet Oxygen Production

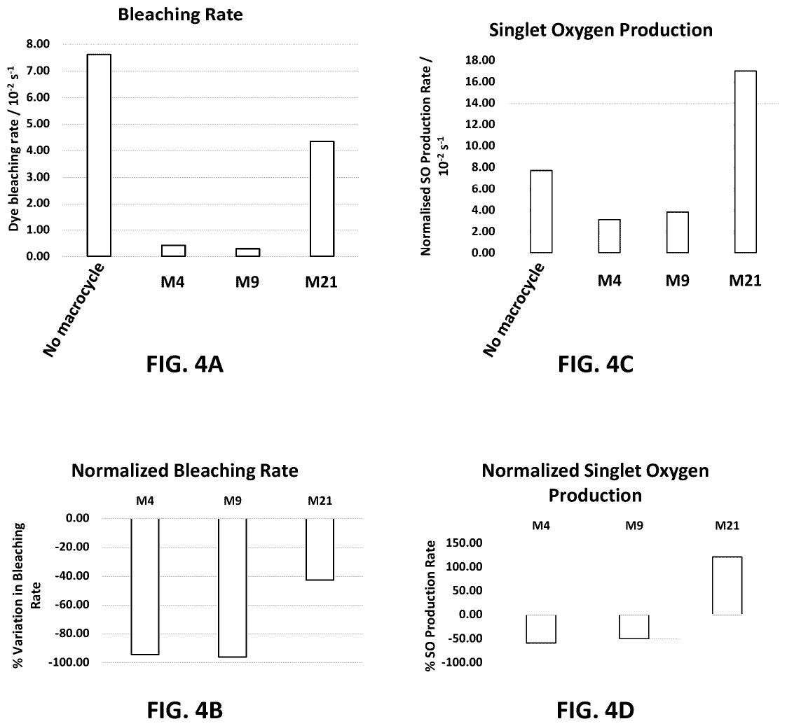

Macrocycles (2-hydroxypropyl)-β-cyclodextrin (M4), methyl-β-cyclodextrin (M9), or cucurbit [7]uril hydrate (M21) were added to solutions containing ffC-LN3-coumarin dye A. These solutions, along with a solution with no added macrocycles were irradiated with a high-power blue LED at different time intervals. This test allowed to calculate the photobleaching rate of ffC-LN3-coumarin dye A once encapsulated within the macrocycles. FIGS. 4A and 4B are bar charts showing the bleaching rates and the normalized bleaching rates for ffC-LN3-coumarin dye A in solution with one of three macrocycles. The results confirm the protection activity of the macrocycles to the dye with respect to the solution without macrocycles. The solutions with (2-hydroxypropyl)-β-cyclodextrin or methyl-β-cyclodextrin experienced a reduction of about 90% of bleaching rate, and the solutions with cucurbit [7]uril hydrate experienced a reduction of about 40% of bleaching rate.

Singlet oxygen can quench fluorescent dye and/or damage nucleotides and dyes. FIGS. 4C and 4D show the results of another test, which was designed to track the production of singlet oxygen, an exemplary ROS, in solutions with (2-hydroxypropyl)-β-cyclodextrin (M4), methyl-β-cyclodextrin (M9), and cucurbit [7]uril hydrate (M21). This test used 9,10-anthracenediyl-bis (methylene) dimalonic acid (ABDA) as a probe molecule for the detection of singlet oxygen. The results in FIGS. 4C and 4D indicate that (2-hydroxypropyl)-β-cyclodextrin and/or methyl-β-cyclodextrin may inhibit the formation of singlet oxygen in solution relative to solution without a macrocycle.

### Example 4. Sequencing with Macrocycle Additives in Scan Mixture

Two macrocycles were selected for inclusion in the scan mixture. Macrocycles (2-hydroxypropyl)-β-cyclodextrin (M4) or methyl-β-cyclodextrin (M9) were added to standard SBS scan mixture at a concentration of 150 mM. No other reagents of the scan mixture were changed. A NextSeq™ 2000 (Illumina) was selected as the sequencing platform, with a blue laser operating at 450 nm wavelength and a green laser operating at 523 nm wavelength. The incorporation mix contained: (1) a set of 3′-AZM blocked nucleotides comprising ffC-LN3-coumarin dye A, ffC-LN3-S07181, ffA-LN3-coumarin dye A, ffA-LN3-NR550S0, ffT-LN3-NR550S0, ffT-LN3-AF550POPOSO (a known green cyanine dye), and pppG (dark G); (2) DNA polymerase Pol 1901; and (3) a glycine buffer. The first 25 cycles used standard scan mixture. Scan mixture with each macrocycle was introduced starting at cycle 26 and used for the following 125 cycles (i.e., through cycle 151).

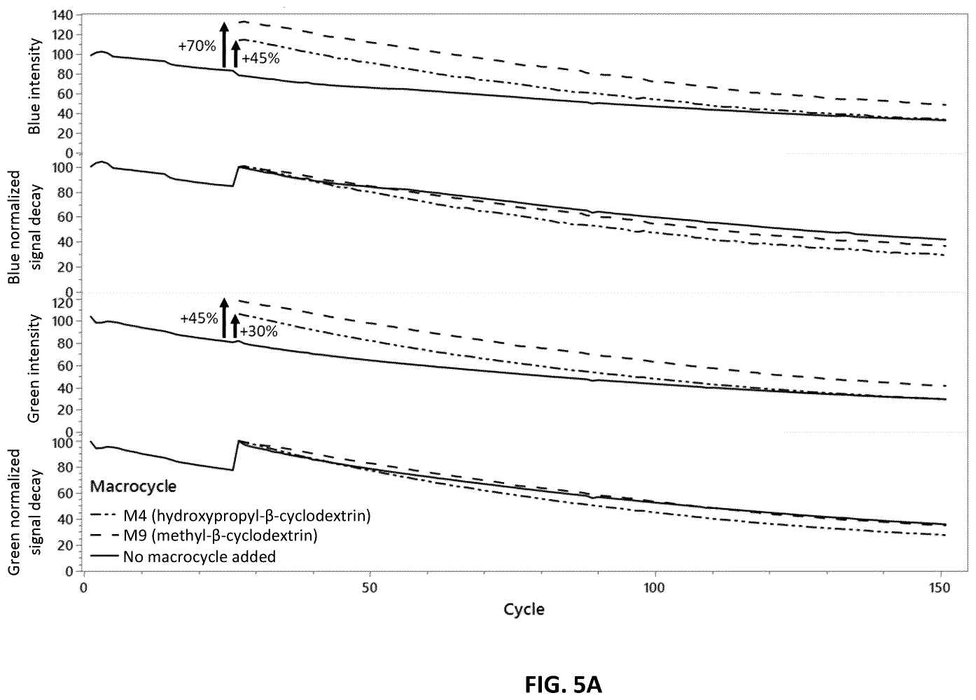

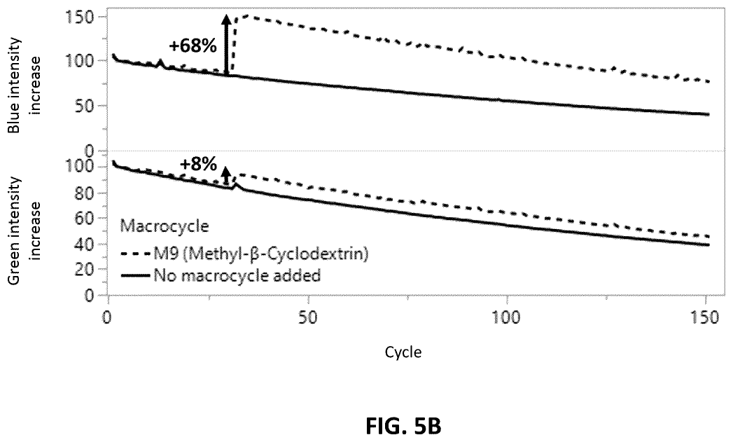

FIG. 5A plots signal intensity and normalized signal decay of the blue and green channels. The results showed that both blue and green fluorescence intensities were impacted by the presence of the macrocycles in the scan mix. The SBS run with (2-hydroxypropyl)-β-cyclodextrin added showed 70% and 45% intensity increases for blue and green, respectively. The SBS run with methyl-β-cyclodextrin showed 45% and 30% intensity increases for blue and green, respectively. In addition, (2-hydroxypropyl)-β-cyclodextrin showed a similar signal decay during 150 cycles of SBS to the SBS run performed without macrocycles (indicated as “No macrocycle added” in FIG. 5A).

In a second SBS experiment, methyl-β-cyclodextrin was added to a standard SBS scan mixture at a concentration of 150 mM. A NextSeq™ 2000 (Illumina) was selected as the sequencing platform, with a blue laser operating at 450 nm wavelength and a green laser operating at 523 nm wavelength. The incorporation mix included: (1) a set of 3′-AOM blocked nucleotides including ffA-DB-AOL-BL-coumarin dye A, ffC-DB-AOL-coumarin dye A, ffC-AOL-NR550SO, ffT-DB-AOL-NR550SO, and pppG (dark G); (2) a DNA polymerase disclosed in U.S. Ser. Nos. 63/412,241 and 63/433,971; and (3) a glycine buffer. The cleavage mixture included a palladium catalyst. The first 31 cycles used a standard scan mixture, without the addition of any macrocycle. 150 mM methyl-β-cyclodextrin was introduced to the standard scan mixture starting at cycle 32 and used for the following 119 cycles (i.e., through cycle 151).

FIG. 5B plots signal intensity of the blue and green channels. The results showed that both blue and green fluorescence intensities were impacted by the presence of the methyl-β-cyclodextrin in the scan mix. The SBS run with methyl-β-cyclodextrin showed 68% and 8% intensity increases for blue and green, respectively. Signal decay was similar to the SBS run performed without macrocycles (indicated as “No macrocycle added” in FIG. 5B).

### Example 5. Methyl-β-Cyclodextrin Concentration Titration

In a first experiment, methyl-β-cyclodextrin concentration titration was performed and included in the reagents were 3′-AZM blocked ffC-sPA-LN3-coumarin dye A and 3′-AZM blocked ffA-LN3-BL-coumarin dye A. Intensity increase was measured by comparing blue intensity of a scan mixture including methyl-β-cyclodextrin with a scan mixture (Tris pH 7.4 (1M), 0.05% Tween 20, sodium ascorbate (20 mM), and hydroxy ethyl gallate (10 mM)). Images were taken with both scan mixes within the same cycle, so enhancement is measured using the same incorporated ffNs. The images were taken at a scanning temperature of 60° C. The process was carried out as follows:
1. Incorporate ffN
2. Wash
3. Add standard scan mixture
4. Generate Image 1
5. Wash
6. Add scan mixture with methyl-β-cyclodextrin
7. Generate Image 2

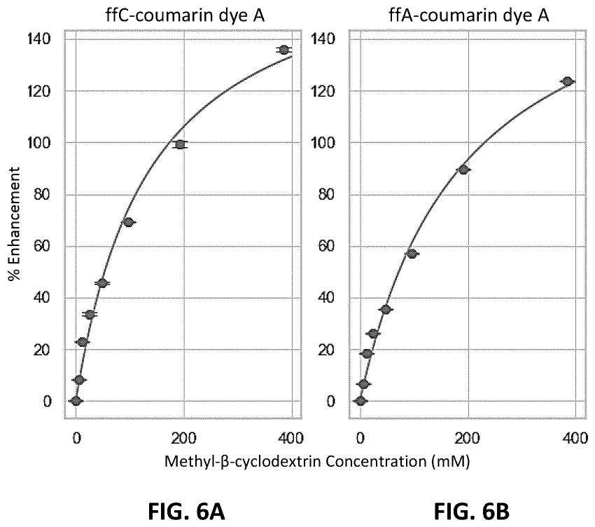

FIGS. 6A and 6B illustrate percentage enhancement of the fluorescent signal as a function of the concentration of methyl-β-cyclodextrin in the scan mixture. Percentage enhancement was calculated in accordance with Equation 1:

$$\\ \begin{matrix}\% Enhancement = \frac{Image 2 intensity - Image 1 intensity}{Image 1 intensity} \times 100 \% . & Equation 1\end{matrix}$$

Both ffC-sPA-LN3-coumarin dye A and ffA-LN3-BL-coumarin dye A reached above 100% enhancement of signal at methyl-β-cyclodextrin concentration of about 400 mM.

In a second experiment, fluorescence intensity as a function of methyl-β-cyclodextrin concentration was measured at three different scanning temperatures at 60° C., 45° C., or 30° C. The ffNs used were 3′-AOM blocked ffA-DB-AOL-BL-coumarin dye A and 3′-AOM blocked ffC-DB-AOL-coumarin dye A.

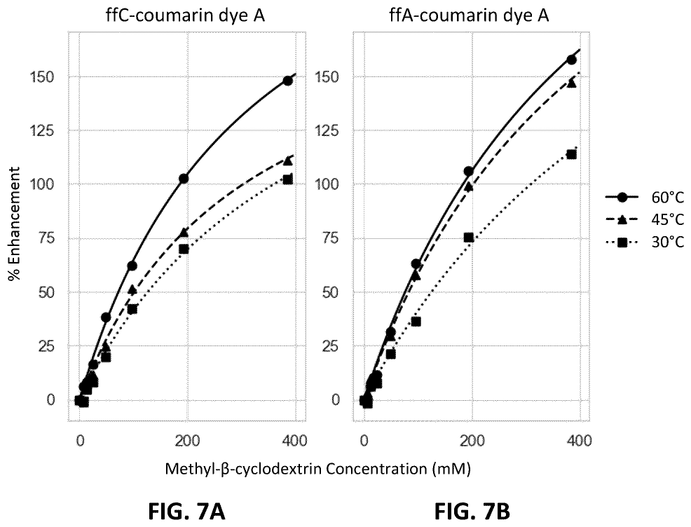

FIGS. 7A and 7B illustrate percentage enhancement of fluorescent signal of the scan mixture with methyl-β-cyclodextrin at 60° C., 45° C. or 30° C. Percentage enhancement was calculated in accordance with Equation 1. For both ffA-DB-AOL-BL-coumarin dye A and ffC-DB-AOL-coumarin dye A, above 100% enhancement of signal was achieved at methyl-β-cyclodextrin concentration of 200 mM at 60° C. and above 100% enhancement of signals were achieved at methyl-β-cyclodextrin concentration of 400 mM at 30° C. and 45° C.

### Example 6. Reduced Intensity Quenching with Macrocycle in Scan Mixture

The effect of different macrocycles on signal quenching was compared on a cBot (Illumina) modified to include a camera for fluorescence imaging. A DNA oligo with a sequence known to cause signal quenching was hybridized to the surface primers. Surface primers were extended using standard Illumina amplification mix, containing a polymerase and dNTPs. The original DNA oligo was then washed off and a sequencing primer hybridized to the extended DNA strand. Cycles of SBS were performed. Included in the incorporation mix was ffC linked to coumarin dye A.

Images were taken in a tris-based scan mixture with the various macrocycles added. The sequence of the incorporated bases was: ACAAAAGGCCTCCTTCATTA (SEQ ID NO. 1). The intensity of ffC-coumarin dye A at cycle 9 was compared to its intensity at cycle 2 (underlined in the incorporated sequence). Without macrocycle added, the intensity at cycle 9 was 61.2% of that at cycle 2. With macrocycle added, the intensity at cycle 9 ranged from 62.7% (M3) to 72.9% (M9). The macrocycles used and their relative intensity decreases are shown in Table 2.

TABLE 2
Cycle 2/Cycle 9 Signal Quenching Comparison

| | Macrocycle | % of Signal Remaining |
|---|---|---|
| Macrocycle | Concentration (mM) | at Quench Site |
| None (Baseline) | N/A | 61.2 |
| M3 - 18 mM | 18 | 62.7 |
| M4 - 150 mM | 150 | 67.5 |
| M9 - 150 mM | 150 | 72.9 |
| M21 - 1 mM | 1 | 64.0 |

### Example 7. Reduced Mismatch Rate Due to Intensity Quenching with Macrocycle in Scan Mixture

The effect of macrocycles on mismatch rate due to intensity quenching was demonstrated on an Illumina NovaSeqX. The sequencing run followed standard protocol. A Human DNA library was used. A sequencing run using the standard scan mix was compared with sequencing runs using scan mixes containing various concentrations of macrocycle M9. The % mismatch at known quenching locations was measured. It was observed that % mismatch at known quenching locations decreased with as M9 concentration increases from 0 to 100 mM, as demonstrated in Table 3.

TABLE 3
Mismatch Rate for Concentrations of M9

| M9 Macrocycle | |
|---|---|
| Concentration (mM) | % Mismatch |
| 0 (Baseline Control) | 10.6 |
| 20 | 7.4 |
| 50 | 4.9 |
| 100 | 3.1 |

1. A method of sequencing a plurality of different target polynucleotides, comprising:
(a) contacting a solid support with an incorporation mixture comprising DNA polymerase and one or more of four different types of nucleotides, wherein the solid support comprises a plurality of different target polynucleotides immobilized thereon, and sequencing primers that are complementary and hybridized to at least a portion of the target polynucleotides;
(b) incorporating one type of nucleotides into the sequencing primers to produce extended copy polynucleotides, wherein one or more of the four types of nucleotides comprises a detectable label; and
(c) imaging and performing one or more fluorescent measurements of the extended copy polynucleotides in an aqueous scan mixture;
wherein the aqueous scan mixture comprises one or more additives for enhancing fluorescent signal intensity of the detectable label, and wherein the one or more additives comprise one or more water-soluble macrocycles.

2. The method of claim 1, wherein the water-soluble macrocycle forms a host-guest complex with the detectable label.

3. The method of claim 1, wherein the water-soluble macrocycle comprises water-soluble cyclodextrins, water-soluble calixarenes, water-soluble cucurbiturils, or optionally substituted analogs, salts, or hydrates thereof, or combination thereof.

4. The method of claim 3, wherein the water-soluble cyclodextrins or the optionally substituted analogs, salts, or hydrates thereof comprise α-cyclodextrin, β-cyclodextrin, γ-cyclodextrin, or substituted analogs, salts or hydrates thereof, or combination thereof.

5. The method of claim 3, wherein the substituted analogs of the water-soluble cyclodextrins are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof.

6. The method of claim 3, wherein the water-soluble cyclodextrin or the substituted analogs, salts, or hydrates thereof are selected from the group consisting of α-cyclodextrin, β-cyclodextrin, (2-hydroxypropl)-β-cyclodextrin, acetyl-β-cyclodextrin, (2-hydroxyethyl)-β-cyclodextrin, heptakis (2,3,6-tri-O-methyl)-β-cyclodextrin, succinyl-β-cyclodextrin, methyl-β-cyclodextrin, carboxymethyl-β-cyclodextrin, heptakis (6-deoxy-6-amino)-β-cyclodextrin heptahydrochloride, γ-cyclodextrin hydrate, (2-hydroxypropyl)-γ-cyclodextrin, and salts and hydrates thereof, and combinations thereof.

7. The method of claim 6, wherein the water-soluble cyclodextrin is (2-hydroxyporpyl)-β-cyclodextrin.

8. The method of claim 6, wherein the water-soluble cyclodextrin is methyl-β-cyclodextrin.

9. The method of claim 3, wherein the concentration of the water-soluble cyclodextrin or the optionally substituted analog, salt, or hydrate thereof in the aqueous scan mixture is from about 1 mM to about 500 mM.

10. The method of claim 3, wherein the binding affinity constant of the water-soluble cyclodextrin to the detectable label is at least about 500M⁻¹.

11. The method of claim 3, wherein the water-soluble cucurbituril or the substituted analogs, salts, or hydrates thereof comprise cucurbituril hydrates or substituted analogs or salts thereof, or combination thereof.

12. The method of claim 11, wherein cucurbituril hydrates are selected from the group consisting of cucuribit [5]uril hydrate, cucuribit [6]uril hydrate, cucuribit [7]uril hydrate, and cucuribit [8]uril hydrate.

13. (canceled)

14. The method of claim 3, wherein the substituted analogs of the water-soluble cucurbiturils are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof.

15. The method of claim 3, wherein the binding affinity constant of the water-soluble cucurbituril to the detectable label is at least about 1×10⁵M⁻¹.

16. The method of claim 3, wherein the concentration of the water-soluble cucurbituril or the optionally substituted analog, salt, or hydrate thereof in the aqueous scan mixture is from about 0.01 mM to about 1 mM.

17. The method of claim 3, wherein the water-soluble calixarenes or the optionally substituted analogs, salts, or hydrates thereof comprise sulfocalixarenes or substituted analogs, salts or hydrates thereof, or combination thereof.

18. The method of claim 3, wherein the substituted analogs of the water-soluble calixarenes are independently substituted with one or more substituents selected from the group consisting of sulfonate, sulfo, hydroxy, carboxy, carboxylate, succinyl, C₁-C₆ alkyl, C₁-C₆ alkyl substituted with sulfo, sulfonate, carboxy, carboxylate, or hydroxy, a hydroxy protecting group, —C(═O) (C₁-C₆ alkyl), —C(═O) CH₃, and —C(═O) Ph, and combinations thereof.

19. The method of claim 3, wherein the water-soluble calixarenes or the optionally substituted analogs, salts, or hydrates thereof are selected from the group consisting of 4-sulfocalix [4] arene, 4-sulfocalix [4] arene hydrate, 4-sulfothiacalix [4] arene, and salts and combinations thereof.

20. The method of claim 3, wherein the binding affinity constant of the water-soluble calixarene to the detectable label is at least about 1000M⁻¹.

21. The method of claim 3, wherein the concentration of the water-soluble calixarene or the optionally substituted analog, salt, or hydrate thereof in the aqueous scan mixture is from about 0.01 mM to about 10 mM.

22.-23. (canceled)

24. A method for enhancing the fluorescence of a fluorescent dye, comprising:
contacting the fluorescent dye with an aqueous scan mixture composition comprising one or more water-soluble macrocycles, wherein the water-soluble macrocycle forms a host-guest complex with the fluorescent dye.

25.-36. (canceled)

37. A kit for use with a sequencing apparatus, comprising:
an incorporation mixture comprising one or more different types of nucleotides, wherein at least one nucleotide is labeled with a detectable label;
an aqueous scan mixture comprising one or more water-soluble macrocycles, wherein the water-soluble macrocycle is capable of encapsulating the detectable label to form a host-guest complex with the detectable label.

38.-50. (canceled)
